SEQUENCE LISTING (13 sequences)

SEQ ID NO: 1 - protein, 281 aa, Artificial Sequence
  synthetic construct; MOG specific scFv
QVQLQQSGAELMKPGASVKISCKATGYTFSSYWIDWVKQRPGHGLEWIGEILPGSGRTNYNEKFKGKTTFTADTSSNTAYIQFSSLTSEDSAVYYCANYGSSRWYFDVWGAGTTVTVSSTKTTAPSVYPLAPVCGDTTGSSVTLGCLVKNSGGGGSGGGGSGGGGSGSSQIVLTQSPAIMSASPGEKVTMTCSASSSISYMHWYQQKPGTSPKRWIYDTSKLASGVPARFSGSGSGTSYSLTISSMEAEDAATYYCHQRSSYPWTFGGGTKLEIKRADAAP

SEQ ID NO: 2 - protein, 241 aa, Artificial Sequence
  synthetic construct; LINGO1 antigen binding domain sequence
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDWYFDLWGRGTLVTVSSGGGGSGGGGSGGGGSDIQMTQSPSFLSASVGDSVAITCRASQDISRYLAWYQQRPGKAPKLLIYDASNLQTGVPSRFSGSGSGTDFTFTITSLQPEDFGTYYCQQYDTLHPSFGPGTTVDIK

SEQ ID NO: 3 - protein, 241 aa, Artificial Sequence
  synthetic construct; LINGO1 antigen binding domain sequence
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDAFDIWGQGTTVTVSSGGGGSGGGGSGGGGSDIQMTQSPATLSLSPGERATLSCRASQSVSSYLAWYQQKPGQAPRLLIYDASNRATGIPARFSGSGSGTDFTLTISSLEPEDFAVYYCQQRSNWPMYTFGQGTKLEIK

SEQ ID NO: 4 - protein, 241 aa, Artificial Sequence
  synthetic construct; LINGO1 antigen binding domain sequence
DIQMTQSPSFLSASVGDSVAITCRASQDISRYLAWYQQRPGKAPKLLIYDASNLQTGVPSRFSGSGSGTDFTFTITSLQPEDFGTYYCQQYDTLHPSFGPGTTVDIKGGGGSGGGGSGGGGSEVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDWYFDLWGRGTLVTVSS

SEQ ID NO: 5 - protein, 241 aa, Artificial Sequence
  synthetic construct; LINGO1 antigen binding site sequence
DIQMTQSPATLSLSPGERATLSCRASQSVSSYLAWYQQKPGQAPRLLIYDASNRATGIPARFSGSGSGTDFTLTISSLEPEDFAVYYCQQRSNWPMYTFGQGTKLEIKGGGGSGGGGSGGGGSEVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDAFDIWGQGTTVTVSS

SEQ ID NO: 6 - protein, 119 aa, Artificial Sequence
  synthetic construct; LINGO1 variable heavy chain
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDWYFDLWGRGTLVTVSS

SEQ ID NO: 7 - protein, 118 aa, Artificial Sequence [sequence omitted]

SEQ ID NO: 8 - protein, 107 aa, Artificial Sequence
  synthetic construct; LINGO1 variable light chain
DIQMTQSPSFLSASVGDSVAITCRASQDISRYLAWYQQRPGKAPKLLIYDASNLQTGVPSRFSGSGSGTDFTFTITSLQPEDFGTYYCQQYDTLHPSFGPGTTVDIK

SEQ ID NO: 9 - protein, 108 aa, Artificial Sequence
  synthetic construct; LINGO1 variable light chain sequence
DIQMTQSPATLSLSPGERATLSCRASQSVSSYLAWYQQKPGQAPRLLIYDASNRATGIPARFSGSGSGTDFTLTISSLEPEDFAVYYCQQRSNWPMYTFGQGTKLEIK

SEQ ID NO: 10 - DNA, 357 bp, Artificial Sequence
  synthetic construct; LINGO1 variable heavy chain sequence
gaagtgcagctgcttgaatctggcggaggactggttcagcctggcggatctctgagactgtcttgtgccgccagcggcttcaccttcagcatctaccctatgttctgggtccgacaggcccctggcaaaggccttgaatgggtgtcctggatcggaccttctggcggcatcaccaaatacgccgacagcgtgaagggcagattcaccatcagccgggacaacagcaagaacaccctgtacctgcagatgaacagcctgagagccgaggacaccgccacctactattgtgccagagagggccacaacgactggtacttcgatctgtggggcagaggcaccctggtcacagtttctagc

SEQ ID NO: 11 - DNA, 354 bp, Artificial Sequence
  synthetic construct; LINGO1 variable heavy chain sequence
gaagtgcagctgcttgaatctggcggaggactggttcagcctggcggatctctgagactgtcttgtgccgccagcggcttcacctttagcgcctacgagatgaaatgggtccgacaggcccctggcaaaggcctggaatgggtgtcagtgatcggacctagcggcggctttaccttctacgccgatagcgtgaagggcagattcaccatcagccgggacaacagcaagaacaccctgtacctgcagatgaacagcctgagagccgaggacaccgccgtgtactattgtgccaccgagggcgacaacgacgccttcgatatttggggccagggcaccaccgtgacagtttctagc

SEQ ID NO: 12 - DNA, 321 bp, Artificial Sequence
  synthetic construct; LINGO1 variable light chain sequence
gatatccagatgactcagagccccagcttcctgtctgccagcgtgggagattctgtggccatcacctgtagagccagccaggacatcagcagatacctggcttggtatcagcagaggcctggcaaggcccctaagctgctgatctacgacgccagcaatctgcagacaggcgtgcccagcagattttctggcagcggcagcggaaccgacttcacctttaccatcaccagcctgcagcctgaggactttggcacctactactgccagcagtacgacacactgcaccctagctttggccctggcaccaccgtggacatcaaa

SEQ ID NO: 13 - DNA, 324 bp, Artificial Sequence
  synthetic construct; LINGO1 variable light chain sequence
gatatccagatgacacagagccctgccacactgtctctgagccctggcgaaagagccacactgagctgtagagccagccagagcgtgtcctcttacctggcctggtatcagcagaagcccggccaagctcctcggctgctgatctacgatgccagcaatagagccacaggcatccccgctagattttccggctctggcagcggcaccgatttcaccctgaccataagcagcctggaacctgaggactttgccgtgtattactgccagcagcggagcaactggcccatgtacacatttggccaggggaccaagctggaaatcaaa

(19) United States
(12) Patent Application Publication
(10) Pub. No.: US 2024/0108653 A1
(43) Pub. Date: Apr. 4, 2024

(54) COMPOSITIONS AND METHODS FOR TREATING WITH CAR CELLS

(71) Applicant: UNIVERSITY OF UTAH RESEARCH FOUNDATION, Salt Lake City, UT (US)

(72) Inventors: Djordje Atanackovic, Salt Lake City, UT (US); Tim Luetkens, Salt Lake City, UT (US); Erin Morales, Salt Lake City, UT (US)

(21) Appl. No.: 18/275,921

(22) PCT Filed: Feb. 4, 2022
(86) PCT No.: PCT/US2022/015307

§ 371 Date: Aug. 4, 2023

Related U.S. Application Data
(60) Provisional application No. 63/146,305, filed on Feb. 5, 2021.

(51) Int. Cl. A61K 35/17; A61K 39/00; A61P 35/00; C07K 14/705; C07K 16/28

(52) U.S. Cl. CPC A61K 35/17; A61K 39/4611; A61K 39/4631; A61P 35/00; C07K 14/705; C07K 16/2803; A61K 2239/15; A61K 2239/17; A61K 2239/21; A61K 2239/22; A61K 2239/28; A61K 2239/46

(57) ABSTRACT

Disclosed are CAR polypeptides comprising a target specific receptor and a death domain. Disclosed are CAR polypeptides comprising a LINGO1 antigen binding domain, a transmembrane domain, and an intracellular signaling domain. Disclosed are CAR cells comprising one or more of the disclosed CAR polypeptides. Disclosed are cells comprising an altered α4β1 integrin. Disclosed are methods of treating comprising administering one or more of the disclosed cells to a subject in need thereof.

### CROSS-REFERENCE TO RELATED APPLICATIONS

This application claims the benefit of U.S. Provisional Patent Application No. 63/146,305, filed on Feb. 5, 2022, which is incorporated by reference herein in its entirety.

### BACKGROUND

Ewing Sarcoma is a solid tumor that is thought to arise from mesenchymal stem cells in pediatric and young adult patients. Treatment for localized Ewing Sarcoma consists of multi-agent neoadjuvant chemotherapy in compressed cycles. This is followed by local control which includes surgery and/or radiation followed by adjuvant chemotherapy. This therapy comes with significant side effects including risks for secondary malignancies, cardiotoxicity and infertility. This confers survival rates of approximately 70%, however in relapsed patients or patients with upfront metastatic disease, overall survival is less than 20%. Current immunotherapy options are limited and still in clinical trials. A randomized phase III trial lead by the Children's Oncology group (COG) studied the use of ganitumab, a monoclonal antibody against Insulin Growth Factor Receptor 1 (IGF1R) in upfront therapy for metastatic Ewing Sarcoma along with conventional chemotherapy. However the study was closed to accrual in the spring of 2019 and ganitumab was discontinued based upon a lack of significant benefit of the addition of ganitumab, as well as the potential for increased toxicity, such as pneumonitis. Furthermore, a clinical trial testing checkpoint inhibitor pembrolizumab in adults with Ewing Sarcoma demonstrated a lack of objective responses. This was attributed to a low mutational burden and lack of PD-L1 expression in Ewing Sarcoma tumors.

Other immune-based therapy options are being explored in adults with Ewing Sarcoma, such as vaccination approaches, which are currently in clinical trials. While cellular immunotherapies using engineered T cells have shown clinical activity in pediatric B cell malignancies, so far it has proven difficult to translate these successes to other types of cancer. These approaches are generally based on the introduction of receptors into the patient's own T cells to redirect them towards tumor cells, with chimeric antigen receptors (CARs) targeting tumor-specific surface antigens. CAR T cells targeting IGF1R and tyrosine kinase-like orphan receptor 1 (ROR1) have been explored in the preclinical setting in Ewing Sarcoma, however these have not yet made it into the clinic and a key problem in the development of effective CAR T cell therapies remains the expression of potential tumor antigens on healthy tissues leading to on-target off-tumor toxicities. To date, no effective immunotherapy has been incorporated into Ewing Sarcoma therapy.

The expression of surface antigen LINGO1 in Ewing sarcoma has been studied. LINGO1 was expressed in over 90% of Ewing Sarcoma tumors and treatment with an antibody-drug conjugate targeting LINGO1 resulted in the efficient killing of Ewing sarcoma cells in vitro. However, while otherwise showing a highly restricted expression in healthy tissues, LINGO1 could also be detected in the central nervous system (CNS), currently prohibiting its use as a therapeutic target. Thus, a therapy designed to target LINGO1 but does not target the CNS is needed.

### BRIEF SUMMARY

Disclosed are CAR polypeptides comprising a target specific receptor and a death domain. For example, disclosed are CAR polypeptides comprising a MOG specific receptor and a Fas domain.

Disclosed are CAR polypeptides comprising a LINGO1 antigen binding domain, a transmembrane domain, and an intracellular signaling domain.

Disclosed are nucleic acid sequences capable of encoding any of the disclosed CAR polypeptides.

Disclosed are cells comprising a CAR, wherein the CAR comprises a target specific receptor and a death domain

Disclosed are cells comprising a CAR, wherein the CAR comprises a LINGO1 antigen binding domain, a transmembrane domain, and an intracellular signaling domain.

Disclosed are cells comprising a first CAR and a second CAR, wherein the first CAR comprises a target specific receptor and a death domain, wherein the second CAR comprises an antigen binding domain, a transmembrane domain, and an intracellular signaling domain.

Disclosed are any of the cells described herein further comprising an altered α4β1 integrin.

Disclosed are compositions comprising one or more of the disclosed CAR polypeptides, CAR cells, CAR nucleic acid sequences, or vectors.

Disclosed are methods of treating a subject having cancer comprising administering a composition comprising a CAR T cell to a subject having cancer, wherein the CAR T cell comprises a CAR polypeptide comprising a LINGO1 antigen binding domain, a transmembrane domain, and an intracellular signaling domain, wherein the subject having cancer has cancer cells expressing LINGO1, wherein the CAR T cell binds LINGO1 on the cancer cells activating the CAR T cell to kill the cancer cell.

Disclosed are methods of treating cancer comprising administering a composition comprising a CAR T cell to a subject having cancer, wherein the CAR T cell comprises a first CAR polypeptide comprising an over-expressed cancer antigen binding domain, a transmembrane domain, and an intracellular signaling domain; and a second CAR polypeptide comprising a non-cancer specific antigen receptor and a death domain; wherein when the CAR T cell binds a non-cancer specific antigen the second CAR polypeptide activates killing of the CAR T cell.

Disclosed are methods of treating Ewing's Sarcoma comprising administering a composition comprising a CAR T cell to a subject having Ewing's Sarcoma, wherein the CAR T cell comprises a first CAR polypeptide comprising a LINGO1 antigen binding domain; and a second CAR polypeptide comprising MOG specific receptor and a Fas domain; wherein upon crossing the blood brain barrier the MOG specific receptor binds MOG on neurons and activates killing of the CAR T cell.

Disclosed are methods of reducing migration of cells across the blood brain barrier comprising administering a composition comprising a cell to a subject wherein the cell comprises an altered α4β1 integrin. In some aspects, the altered α4β1 integrin can be any of those described herein.

Disclosed are methods of inducing apoptosis of a CAR T cell comprising administering a composition comprising a CAR T cell to a subject wherein the CAR T cell comprises a CAR polypeptide comprising a target specific receptor and a death domain, wherein when the target specific antigen receptor of the CAR T cell binds the target, the death domain activates killing of the CAR T cell.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a schematic of T cell engineering approach. CAR T cells targeting LINGO1 expressing a CNS-sensing suicide switch recognizing MOG and lacking ITGA4/ITGB1 to prevent VCAM1 adhesion and blood-brain barrier migration.

FIGS. 2A, 2B and 2C show antigen expression in Ewing Sarcoma. (FIG. 2A) mRNA expression of tumor antigen LINGO1 and the brain-specific surface antigen MOG was determined in Ewing sarcoma cell lines as well as healthy brain cDNA using RT-PCR. (FIG. 2B) Schematic of LINGO1-specific CAR construct. (FIG. 2C) Killing of K562 cells engineered to express luciferase and LINGO1 by mock transduced T cells (Ctrl) or T cells expressing two different CAR constructs targeting LINGO1 (Li6 or Li8) at an effector-target ratio of 0.33:1.

FIGS. 3A-3G show development of integrin knockout and miFas receptors. (FIG. 3A) Schematic drawing of activated a4b1 integrin on T cells binding to VCAM1 on brain endothelial cells. (FIG. 3B) Surface ITGA4 and ITGB1 expression on Jurkat cells after transduction with single guide RNAs targeting the respective integrin or a control guide RNA. (FIG. 3C) Adhesion of Jurkat cells expressing firefly luciferase to immobilized recombinant VCAM1 after 2 washes with PBS using an automated plate washer. Remaining cells were quantified using a luminescence plate reader. (FIG. 3D) Schematic drawing of miFas constructs using antigen-specific scFv domains, monomeric (HLAA2 and LNGFR) or dimeric (CD8) hinge domains, two chemically inducible dimerization domains, and the intracellular Fas signaling domain. (FIG. 3E) Expansion of human donor T cells transduced with the different miFas constructs after flow cytometry sorting. (FIG. 3F) 3-(4,5-Dimethylthiazol-2-yl)-2,5-diphenyltetrazolium bromide (MTT) assay to determine killing of T cell line J76 expressing miFas constructs after treatment with 20 nM AP20187 (chemical inducer of FKBP-mediated dimerization). (FIG. 3G) Luciferase-based cytotoxicity assay to determine killing of J76 cells expressing miFas constructs targeting CD19 after 24 h co-culture with K562 cells engineered to express CD19.

FIGS. 4A-4I show LINGO1-specific CAR T cells show cytotoxic activity against Ewing sarcoma. (FIG. 4A) mRNA expression of LINGO1 and GAPDH housekeeping gene in Ewing sarcoma cell lines and healthy brain tissue as determined by RT-PCR. (FIG. 4B) Schema of CAR construct and four LINGO1-specific scFv domains (FIG. 4C) Binding of LINGO1-specific scFvs at 5 g/ml and secondary staining with an anti-FLAG/APC antibody (clone: L5). against Ewing sarcoma cell lines as determined by flow cytometry. Secondary antibody alone is shown in grey. (FIG. 4D) LINGO1 CAR surface expression on primary T cells after staining with an anti-HA/APC antibody as determined by flow cytometry. (FIG. 4E) IFNγ secretion as determined by ELISA and (FIG. 4F) anti-tumor activity as determined by luminescence-based cytotoxicity assay of CAR T cells expressing LINGO1 CAR variants after 24 h co-culture with A673 cells at an effector-target ratio of 1:1. (FIG. 4G) Expansion of LINGO1 CAR T cells based on Li81 VLVH and T cels expressing a CAR without a binding domain (ΔscFv). (FIG. 4H) IFNγ and IL2 production as determined by ELISA and (FIG. 4I) anti-tumor activity as determined by luminescence-based cytotoxicity assay by LINGO1 CAR T cells after 24 h co-culture with Ewing sarcoma cell lines at different effector-target ratios. Data represent mean±SD from 3 technical replicates. Statistical significance was determined by two-sided Student's t test.

FIGS. 5A-5H show knockout of ITGA4 prevents BBB migration of CAR T cells but does not alter anti-tumor activity. (FIG. 5A) Schema of the active integrin α4β1 heterodimer binding to the endothelial adhesion protein VCAM1. (FIG. 5B) Exon structure of integrins a4 and β1 and gRNA sequences. SP signal peptide; EC extracellular domain; TM transmembrane domain; IC intracellular domain. (FIG. 5C) ITGA4 and ITGB1 expression as determined by flow cytometry on the surface of Jurkat T cells expressing constitutively active ITGA4/ITGB1 after lentiviral transduction with Cas9 and individual gRNAs against ITGA4 (sgITGA4), ITGAB1 (sgITGB1), or a negative control gRNA (sgCtrl) before and after enrichment by fluorescence-activated cell sorting. (FIG. 5D) Adhesion of Jurkat cells expressing firefly luciferase transduced with different gRNA constructs to recombinant VCAM1 after 2 washes with 300 μl PBS at 200 μl/s using an automated plate washer as determined by luminescence assay. (FIG. 5E) Schema of in vivo experiment to determine BBB migration of Jurkat cells transduced with sgITGA4 or sgCtrl. Five NOD.Cg-Rag1^{tm1Mom}Il2rg^{tm1Wjl}/SzJ (NRG) mice per group were injected with 2×10⁶ luciferase-expressing Jurkat cells by tail vein and brain were harvested on day 29. In vivo imaging system (IVIS) data for animals on day 24 h after injection with luciferase-expressing Jurkat cells. Dotted lines indicate regions of interest to differentiate between “head” and “body” areas. (FIG. 5F) Quantification of average radiance per animal shown in panel E from “head” and “body” areas as well as (FIG. 5G) explanted brains. (FIG. 5H) Left Representative staining with anti-huCD45/APC and anti-CD3/FITC. Right Quantification of human T cell numbers in lymphocyte populations isolated from explanted brains as determined by flow cytometry. Data represent mean±SD from 3 technical replicates. Statistical significance was determined by two-sided Student's t test. ns not significant. IVIS quantification was carried out using LivinImage software (Perkin-Elmer).

FIGS. 6A-6F show ITGA4^{ko} LINGO1 CAR T cells can be manufactured and show increased anti-tumor activity in vitro and in vivo. (FIG. 6A) Schema of ITGA4^{ko} LINGO1 CAR T cell manufacturing process. (FIG. 6B) ITGA4 and CAR expression levels on ITGA4^{ko} LINGO1 CAR T cells as determined by flow cytometry. (FIG. 6C) Expression of ITGA4 over time after gRNA/Cas9 electroporation as determined by flow cytometry. (FIG. 6D) Expansion of ITGA4^{ko} T cells during production. (FIG. 6E) Adhesion of primary human T cells after ITGA4 knockout to recombinant VCAM1 after 3 washes with PBS as determined by flow cytometry. (FIG. 6F) Anti-tumor activity of ITGA4^{ko} LINGO1 CAR T cells against Ewing sarcoma cell line A673 as determined by luminescence-based cytotoxicity assay.

FIGS. 7A-7E show that reduced expansion of LINGO1 CAR T cells is rescued by c-Jun overexpression while maintaining anti-tumor activity. FIG. 7A shows CAR T cells targeting LINGO1 using the Li81 scFv show reduced expansion during CAR T cell production compared to T cells expressing a CAR without a binding domain. FIG. 7B shows a construct comprising c-Jun and CAR. FIG. 7C shows expressing the CAR together with the full-length sequence of human c-Jun rescues expansion. LINGO1 CAR T cells expressing c-Jun maintain their FIG. 7D shows anti-tumor activity as well as FIG. 7E showing effector cytokine expression in an overnight co-culture cytotoxicity assay targeting the Ewing sarcoma cell line A673 with or without ITGA4 knockout at different effector-target ratios.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a polypeptide” includes a plurality of such polypeptides, reference to “the cell” is a reference to one or more cells and equivalents thereof known to those skilled in the art, and so forth.

As used herein, “target specific receptor” means a receptor or binding domain that interacts or binds with a target of interest. In some aspects, the target of interest is present on a non-disease of interest cell/tissue but is not present on a cell having a disease of interest. A non-disease of interest cell/tissue can be a healthy cell/tissue or can be a cell having any disease that is not the disease of interest. A disease of interest is the disease currently being treated. For example, a target specific receptor can interact or bind to a target present on healthy cell but is not present on a cancer cell.

As used herein, “death domain” means a polypeptide which induces killing of a cell (e.g. CAR T cell) upon binding of the target specific receptor to its target antigen.

A “single-chain variable fragment (scFv)” means a protein comprising the variable regions of the heavy and light chains of an antibody. A scFv can be a fusion protein comprising a variable heavy chain, a linker, and a variable light chain.

A “fragment antigen-binding fragment (Fab)” is a region of an antibody that binds to antigen. A Fab comprises constant and variable regions from both heavy and light chains.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the terms “treatment,” “treating,” and the like, refer to obtaining a desired pharmacologic and/or physiologic effect. In some aspects, “treat” is meant to mean administer a CAR T cell or composition described herein to a subject, such as a human or other mammal (for example, an animal model), that has a disease or condition (e.g. Ewing's Sarcoma or another type of cancer), in order to prevent or delay a worsening of the effects of the disease or condition, or to partially or fully reverse the effects of the disease or condition. In some aspects, the disease or condition can be cancer. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some embodiments, treatment comprises delivery of one or more of the disclosed CAR T cells or compositions to a subject.

As used herein, “prevent” is meant to mean minimize the chance that a subject who has an increased susceptibility for developing disease, disorder or condition will develop the disease, disorder or condition (e.g. Ewing's Sarcoma or another type of cancer). For example, prevent as used herein can mean minimize the chance that a subject who has an increased susceptibility for developing cancer will develop it.

As used herein, the terms “administering” and “administration” refer to any method of providing a disclosed polypeptide, polynucleotide, vector, composition, or a pharmaceutical preparation to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed conjugate so as to treat a subject or induce apoptosis. In an aspect, the skilled person can also alter or modify an aspect of an administering step so as to improve efficacy of a disclosed polypeptide, polynucleotide, vector, composition, or a pharmaceutical preparation.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “polypeptide,” “peptide,” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. CAR Polypeptides

Disclosed herein are CAR polypeptides. As used herein, the term “CAR polypeptide” can be used interchangeably with “CAR”. In some aspects, the disclosed CARs can function as a suicide switch that can be used to initiate apoptosis of a cell comprising the CAR. In some aspects, the disclosed CARs bind to a specific target on a cell type of interest.

1. Suicide Switch

Disclosed are CARs comprising a target specific receptor and a death domain.

In some aspects, the disclosed CARs comprising a target specific receptor and a death domain can be also be referred to as suicide switches. The disclosed CARs can be known as suicide switches because, in some aspects, the disclosed CARs comprising a target specific receptor and a death domain can initiate apoptosis of a cell comprising the CAR.

i. Target Specific Receptor

In some aspects, the target specific receptor can be, but is not limited to, an antibody or antigen binding fragment of an antibody (e.g. Fab or single chain variable fragment (scFv)). In some instances, the scFv, comprising both a heavy chain variable region and the light chain variable region, can comprise the N-terminal region of the heavy chain variable region linked to the C-terminal region of the light chain variable region. In some instances, the scFv comprises the C-terminal region of the heavy chain variable region linked to the N-terminal region of the light chain variable region.

In some aspects, the target specific receptor interacts or binds with a target found only in the central nervous system. In some aspects, a target can be any membrane protein strongly and widely expressed in the brain, preferably on glial cells rather than neurons, and absent from any other tissues. For example, a target found only in the central nervous system can be, but is not limited to, myelin oligodendrocyte glycoprotein (MOG), myelin basic protein (MBP), growth associated protein 43 (GAP43), EPH receptor B1 (EPHB1), and L1CAM. In some aspects, the target specific receptor can be a MOG specific receptor. In some aspects, a MOG specific receptor can be a MOG specific scFv. For example, a MOG specific scFv can have the amino acid sequence comprising

(SEQ ID NO: 1)
QVQLQQSGAELMKPGASVKISCKATGYTFSSYWIDWVKQRPGHGLEWIG
EILPGSGRTNYNEKFKGKTTFTADTSSNTAYIQFSSLTSEDSAVYYCAN
YGSSRWYFDVWGAGTTVTVSSTKTTAPSVYPLAPVCGDTTGSSVTLGCL
VKNSGGGGSGGGGSGGGGSGSSQIVLTQSPAIMSASPGEKVTMTCSASS
SISYMHWYQQKPGTSPKRWIYDTSKLASGVPARFSGSGSGTSYSLTISS
MEAEDAATYYCHQRSSYPWTFGGGTKLEIKRADAAP.

ii. Death Domain

In some aspects, the death domain can be any known death domain that triggers, signals, or initiates cell death, particularly through apoptosis. In some aspects, the death domain can be a Fas domain, tumor necrosis factor receptor 1 (TNFR1) domain, death receptor 3 (DR3) domain, death receptor 4 (DR4) domain, death receptor 5 (DR5) domain, fas-associated death domain (FADD) domain, or a caspase.

In some aspects, the target specific receptor and the death domain are conjugated to each other. In some aspects, the target specific receptor and the death domain form a fusion protein.

iii. Other Domains

In some aspects, the disclosed CARs comprising a target specific receptor and a death domain further comprise a transmembrane domain between the target specific receptor and the death domain. In some aspects, the disclosed CARs comprising a target specific receptor and a death domain further comprise a hinge region between the target specific receptor and the death domain. In some aspects, the disclosed CARs comprising a target specific receptor and a death domain further comprise a transmembrane domain and a hinge region between the target specific receptor and the death domain. In some aspects, the target specific receptor is conjugated to the hinge region, the hinge region is conjugated to the transmembrane domain, and the transmembrane domain is conjugated to the death domain.

In some aspects, the hinge region can be monomeric. In some aspects, the monomeric hinge region is HLA-A2. In some aspects, the HLA-A2 can lack the very polymorphic alpha1 and alpha2 domains to prevent potential targeting of the hosts T cells to a CAR T cell. In some aspects, the hinge region is an extracellular domain of the same protein the transmembrane domain is generated from. In some aspects, target specific receptor, hinge region, transmembrane domain and death domain are all from different parent proteins.

In some instances, the hinge region can be located between the target specific receptor and the death domain. In some instances, the hinge region allows for the target specific receptor to bind to the target. For example, the hinge region can increase the distance of the target specific receptor to the cell surface and provide flexibility.

2. LINGO Specific CAR

Disclosed are CAR polypeptides comprising a Leucine-Rich Repeat and Ig Domain Protein 1 (LINGO1) antigen binding domain, a transmembrane domain, and an intracellular signaling domain.

i. LINGO1 Antigen Binding Domain

In some instances, the LINGO1 antigen binding domain can be an antibody fragment or an antigen-binding fragment that specifically binds to LINGO1. In some instances, the LINGO1 antigen binding domain can be any recombinant or engineered protein domain capable of binding LINGO1.

In some instances, the LINGO1 antigen binding domain can be a Fab or a single-chain variable fragment (scFv) of an antibody that specifically binds LINGO1. In some instances, the scFv, comprising both the heavy chain variable region and the light chain variable region, can comprise the N-terminal region of the heavy chain variable region linked to the C-terminal region of the light chain variable region. In some instances, the scFv comprises the C-terminal region of the heavy chain variable region linked to the N-terminal region of the light chain variable region.

In some instances, the LINGO1 antigen binding domain comprises an amino acid sequence set forth in SEQ ID NO: 2, 3, 4, or 5. In some instances, the LINGO1 antigen binding domain can comprise a heavy chain van able region, a light chain variable region, and a linker that links the heavy chain variable region to the light chain variable region. For example, SEQ ID NOs: 2-5 comprise the heavy chain variable region, linker, and light chain variable region (see Table 1). In some instances, the linker can be directly involved in the binding of LINGO1 to the LINGO1 antigen binding domain. In some instances, the linker can be indirectly involved in the binding of LINGO1 to the LINGO1 antigen binding domain.

TABLE 1
Examples of LINGO1 antigen binding
domain sequences. Variable heavy chain (bold),
linker (underlined), and variable light chain

| | |
|---|---|
| SEQ ID NO: 2 | EVQLLESGGGLVQPGGSLRLSCAASGFTFSIYPM |
| Li62 | FWVRQAPGKGLEWVSWIGPSGGITKYADSVKGRF |
| VHVL | TISRDNSKNTLYLQMNSLRAEDTATYYCAREGHN |
| orientation | DWYFDLWGRGTLVTVSSGGGGSGGGGSGGGGSDI |
| | QMTQSPSFLSASVGDSVAITCRASQDISRYLAWY |
| | QQRPGKAPKLLIYDASNLQTGVPSRFSGSGSGTD |
| | FTFTITSLQPEDFGTYYCQQYDTLHPSFGPGTTV |
| | DIK |
| SEQ ID NO: 3 | EVQLLESGGGLVQPGGSLRLSCAASGFTFSAYEM |
| Li81 | KWVRQAPGKGLEWVSVIGPSGGFTFYADSVKGRF |
| VHVL | TISRDNSKNTLYLQMNSLRAEDTAVYYCATEGDN |
| orientation | DAFDIWGQGTTVTVSSGGGGSGGGGSGGGGSDIQ |
| | MTQSPATLSLSPGERATLSCRASQSVSSYLAWYQ |
| | QKPGQAPRLLIYDASNRATGIPARFSGSGSGTDF |
| | TLTISSLEPEDFAVYYCQQRSNWPMYTFGQGTKL |
| | EIK |
| SEQ ID NO: 4 | DIQMTQSPSFLSASVGDSVAITCRASQDISRYLA |
| Li62 | WYQQRPGKAPKLLIYDASNLQTGVPSRFSGSGSG |
| VLVH | TDFTFTITSLQPEDFGTYYCQQYDTLHPSFGPGT |
| orientation | TVDIKGGGGSGGGGSGGGGSEVQLLESGGGLVQP |
| | GGSLRLSCAASGFTFSIYPMFWVRQAPGKGLEWV |
| | SWIGPSGGITKYADSVKGRFTISRDNSKNTLYLQ |
| | MNSLRAEDTATYYCAREGHNDWYFDLWGRGTLVT |
| | VSS |
| SEQ ID NO: 5 | DIQMTQSPATLSLSPGERATLSCRASQSVSSYLA |
| Li81 | WYQQKPGQAPRLLIYDASNRATGIPARFSGSGSG |
| VLVH | TDFTLTISSLEPEDFAVYYCQQRSNWPMYTFGQG |
| orientation | TKLEIKGGGGSGGGGSGGGGSEVQLLESGGGLVQ |
| | PGGSLRLSCAASGFTFSAYEMKWVRQAPGKGLEW |
| | VSVIGPSGGFTFYADSVKGRFTISRDNSKNTLYL |
| | QMNSLRAEDTAVYYCATEGDNDAFDIWGQGTTVT |
| | VSS |

In some instances, the LINGO1 antigen binding domain comprises a variable heavy chain comprising a sequence having at least 90% identity to a sequence set forth in SEQ ID NOs: 6 or 7 (See Table 2). In some instances, the LINGO1 antigen binding domain comprises a variable heavy chain comprising a sequence having at least 90, 91, 92, 93, 94, 95, 96, 97, 98, 99, or 100% identity to a sequence set forth in SEQ ID NOs: 6 or 7.

TABLE 2
Examples of LINGO1 variable heavy chain sequences

| | |
|---|---|
| SEQ ID NO: 6 | EVQLLESGGGLVQPGGSLRLSCAASGFTFSIYPM |
| | FWVRQAPGKGLEWVSWIGPSGGITKYADSVKGRF |
| | TISRDNSKNTLYLQMNSLRAEDTATYYCAREGHN |
| | DWYFDLWGRGTLVTVSS |
| SEQ ID NO: 7 | EVQLLESGGGLVQPGGSLRLSCAASGFTFSAYEM |
| | KWVRQAPGKGLEWVSVIGPSGGFTFYADSVKGRF |
| | TISRDNSKNTLYLQMNSLRAEDTAVYYCATEGDN |
| | DAFDIWGQGTTVTVSS |

In some instances, the LINGO1 antigen binding domain comprises a variable light chain comprising a sequence having at least 90% identity to a sequence set forth in SEQ ID NOs: 8 or 9 (See Table 2). In some instances, the LINGO1 antigen binding domain comprises a variable light chain comprising a sequence having at least 90, 91, 92, 93, 94, 95, 96, 97, 98, 99, or 100% identity to a sequence set forth in SEQ ID NOs: 8 or 9.

TABLE 3
Examples of LINGO1 variable light chain sequences

| | |
|---|---|
| SEQ ID NO: 8 | DIQMTQSPSFLSASVGDSVAITCRASQDISRYLA |
| | WYQQRPGKAPKLLIYDASNLQTGVPSRFSGSGSG |
| | TDFTFTITSLQPEDFGTYYCQQYDTLHPSFGPGT |
| | TVDIK |
| SEQ ID NO: 9 | DIQMTQSPATLSLSPGERATLSCRASQSVSSYLA |
| | WYQQKPGQAPRLLIYDASNRATGIPARFSGSGSG |
| | TDFTLTISSLEPEDFAVYYCQQRSNWPMYTFGQG |
| | TKLEIK |

ii. Transmembrane Domain

In some aspects, the transmembrane domain comprises a CD8α domain, CD3ζ, FcεR1γ, CD4, CD7, CD28, OX40, or a H2-Kb domain.

In some aspects, the transmembrane domain comprises an immunoglobulin Fe domain. In some instances, the immunoglobulin Fe domain can be an immunoglobulin G Fc domain.

In some aspects, the transmembrane domain is located between the LINGO1 antigen binding domain and the intracellular signaling domain.

iii. Intracellular Signaling Domain

In some aspects, the intracellular signaling domain comprises a co-stimulatory signaling region.

In some aspects, the co-stimulatory signaling region comprises the cytoplasmic domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

In some aspects, the intracellular signaling domain is a T cell signaling domain. For example, in some aspects, the intracellular signaling domain comprises a CD3 zeta (CD3ζ) signaling domain.

In some aspects, the intracellular signaling domain comprises a CD3ζ signaling domain and a co-stimulatory signaling region, wherein the co-stimulatory signaling region comprises the cytoplasmic domain of CD28 or 4-1BB.

iv. Hinge

In some aspects, the disclosed CARs further comprise a hinge region. For example, disclosed are CAR polypeptides comprising a LINGO1 antigen binding domain, a transmembrane domain, an intracellular signaling domain, and a hinge region.

In some aspects, the hinge region is located between the LINGO1 antigen binding domain and the transmembrane domain.

In some aspects, the hinge region allows for the LINGO1 antigen binding domain to bind to the antigen (LINGO1). For example, the hinge region can increase the distance of the binding domain to the cell surface and provide flexibility.

In some aspects, the hinge region is an extracellular portion of the same protein from which the transmembrane domain is generated.

v. Tag

In some aspects, the disclosed CARs further comprise a tag sequence. For example, disclosed are CAR polypeptides comprising a LINGO1 antigen binding domain, a transmembrane domain, an intracellular signaling domain, and a tag sequence.

In some aspects, the tag sequence is located between the LINGO1 antigen binding domain and the transmembrane domain.

In some aspects, the tag sequence can be any sequence used to detect or label the CAR. In some aspects, the tag sequence is a hemagglutinin tag. In some aspects, the tag sequence can be, but is not limited to, FLAG, hemagglutinin, myc, or strep tag. In some aspects, the tag sequence can be used to detect the CAR on the surface of a cell but it can also be used to stimulate the cells during production or once in the patient.

### C. CAR Nucleic Acid Sequences

Disclosed are nucleic acid sequences capable of encoding any of the disclosed CAR polypeptides (also referred to herein as a “CAR nucleic acid sequence”. For example, disclosed are nucleic acid sequences capable of encoding a CAR polypeptide comprising a LINGO1 antigen binding domain, a transmembrane domain, and an intracellular signaling domain. Also disclosed are nucleic acid sequences capable of encoding a CAR comprising a target specific receptor and a death domain

In some aspects, a nucleic acid sequence encoding the LINGO1 antigen binding domain comprises a sequence that encodes a variable heavy chain having at least 90% identity to a sequence set forth in SEQ ID NOs: 6 or 7. In some instances, the LINGO1 antigen binding domain comprises a sequence that encodes a variable heavy chain having at least 90, 91, 92, 93, 94, 95, 96, 97, 98, 99, or 100% identity to a sequence set forth in SEQ ID NOs: 6 or 7.

In some aspects, a nucleic acid sequence encoding the LINGO1 antigen binding domain comprises a variable heavy chain sequence having at least 90% identity to a sequence set forth in SEQ ID NOs: 10 or 11. In some instances, the LINGO1 antigen binding domain comprises a variable heavy chain having at least 90, 91, 92, 93, 94, 95, 96, 97, 98, 99, or 100% identity to a sequence set forth in SEQ ID NOs: 10 or 11.

TABLE 4
Examples of LINGO1 variable heavy chain sequences

| | |
|---|---|
| SEQ ID NO: 10 | GAAGTGCAGCTGCTTGAATCTGGCGGAGGACTG |
| | GTTCAGCCTGGCGGATCTCTGAGACTGTCTTGT |
| | GCCGCCAGCGGCTTCACCTTCAGCATCTACCCT |
| | ATGTTCTGGGTCCGACAGGCCCCTGGCAAAGGC |
| | CTTGAATGGGTGTCCTGGATCGGACCTTCTGGC |
| | GGCATCACCAAATACGCCGACAGCGTGAAGGGC |
| | AGATTCACCATCAGCCGGGACAACAGCAAGAAC |
| | ACCCTGTACCTGCAGATGAACAGCCTGAGAGCC |
| | GAGGACACCGCCACCTACTATTGTGCCAGAGAG |
| | GGCCACAACGACTGGTACTTCGATCTGTGGGGC |
| | AGAGGCACCCTGGTCACAGTTTCTAGC |
| SEQ ID NO: 11 | GAAGTGCAGCTGCTTGAATCTGGCGGAGGACTG |
| | GTTCAGCCTGGCGGATCTCTGAGACTGTCTTGT |
| | GCCGCCAGCGGCTTCACCTTTAGCGCCTACGAG |
| | ATGAAATGGGTCCGACAGGCCCCTGGCAAAGGC |
| | CTGGAATGGGTGTCAGTGATCGGACCTAGCGGC |
| | GGCTTTACCTTCTACGCCGATAGCGTGAAGGGC |
| | AGATTCACCATCAGCCGGGACAACAGCAAGAAC |
| | ACCCTGTACCTGCAGATGAACAGCCTGAGAGCC |
| | GAGGACACCGCCGTGTACTATTGTGCCACCGAG |
| | GGCGACAACGACGCCTTCGATATTTGGGGCCAG |
| | GGCACCACCGTGACAGTTTCTAGC |

In some aspects, a nucleic acid sequence encoding the LINGO1 antigen binding domain comprises a sequence that encodes a variable light chain having at least 90% identity to a sequence set forth in SEQ ID NOs: 8 or 9. In some instances, the LINGO1 antigen binding domain comprises a sequence that encodes a variable light chain having at least 90, 91, 92, 93, 94, 95, 96, 97, 98, 99, or 100% identity to a sequence set forth in SEQ ID NOs: 8 or 9.

In some aspects, a nucleic acid sequence encoding the LINGO1 antigen binding domain comprises a variable light chain sequence having at least 90% identity to a sequence set forth in SEQ ID NOs: 12 or 13. In some instances, the LINGO1 antigen binding domain comprises a variable light chain sequence having at least 90, 91, 92, 93, 94, 95, 96, 97, 98, 99, or 100% identity to a sequence set forth in SEQ ID NOs: 12 or 13.

TABLE 5
Examples of LINGO1 variable light chain sequences

| | |
|---|---|
| SEQ ID NO: 12 | GATATCCAGATGACTCAGAGCCCCAGCTTCCTG |
| | TCTGCCAGCGTGGGAGATTCTGTGGCCATCACC |
| | TGTAGAGCCAGCCAGGACATCAGCAGATACCTG |
| | GCTTGGTATCAGCAGAGGCCTGGCAAGGCCCCT |
| | AAGCTGCTGATCTACGACGCCAGCAATCTGCAG |
| | ACAGGCGTGCCCAGCAGATTTTCTGGCAGCGGC |
| | AGCGGAACCGACTTCACCTTTACCATCACCAGC |
| | CTGCAGCCTGAGGACTTTGGCACCTACTACTGC |
| | CAGCAGTACGACACACTGCACCCTAGCTTTGGC |
| | CCTGGCACCACCGTGGACATCAAA |
| SEQ ID NO: 13 | GATATCCAGATGACACAGAGCCCTGCCACACTG |
| | TCTCTGAGCCCTGGCGAAAGAGCCACACTGAGC |
| | TGTAGAGCCAGCCAGAGCGTGTCCTCTTACCTG |
| | GCCTGGTATCAGCAGAAGCCCGGCCAAGCTCCT |
| | CGGCTGCTGATCTACGATGCCAGCAATAGAGCC |
| | ACAGGCATCCCCGCTAGATTTTCCGGCTCTGGC |
| | AGCGGCACCGATTTCACCCTGACCATAAGCAGC |
| | CTGGAACCTGAGGACTTTGCCGTGTATTACTGC |
| | CAGCAGCGGAGCAACTGGCCCATGTACACATTT |
| | GGCCAGGGGACCAAGCTGGAAATCAAA |

In some aspects, any of the disclosed nucleic acid sequences can further comprise a sequence that encodes c-Jun.

### D. CAR Cells

Disclosed are cells comprising any of the disclosed CAR polypeptides, CAR nucleic acids, or disclosed vectors. These cells can be considered genetically modified.

In some instances, the cell can be a T cell. For example, T cell can be a CD8+ T cell. In some instances, the can be a human cell. In some aspects, the cell can be a natural killer cell.

1. Suicide Switch CAR

Disclosed are cells comprising any of the disclosed suicide switches. For example, disclosed are cells comprising a CAR, wherein the CAR comprises a target specific receptor and a death domain. As described herein, in some aspects, the disclosed suicide switches further comprise one or more of a transmembrane domain and hinge region.

In some aspects, the target specific receptor and death domain can be any of those disclosed herein. For example, the target specific receptor can be a MOG specific receptor and the death domain can be a Fas domain. In some aspects, the target specific receptor can be a MBP, GAP43, EPHB1, or L1CAM.

In some aspects, the target specific receptor is completely or partially located on the extracellular surface of the CAR cell. In some aspects, the death domain is completely or partially located on the intracellular surface of the CAR cell. In some aspects, the transmembrane domain is completely or partially located in the cell membrane of the CAR cell. In some aspects, the hinge region is completely or partially located on the extracellular surface of the CAR cell.

2. LINGO CAR

Disclosed are cells comprising any of the disclosed LINGO specific CARs. For example, disclosed are cells comprising a CAR, wherein the CAR comprises a LINGO1 antigen binding domain, a transmembrane domain, and an intracellular signaling domain.

In some aspects, the LINGO1 antigen binding domain, transmembrane domain, and intracellular signaling domain can be any of those disclosed herein.

3. Suicide Switch CAR Plus Second CAR

Disclosed are cells comprising any of the disclosed suicide switches and a second CAR. Disclosed are cells comprising one or more of the disclosed CARs. For example, disclosed are cells comprising a first CAR and a second CAR, wherein the first CAR comprises a target specific receptor and a death domain, wherein the second CAR comprises an antigen binding domain, a transmembrane domain, and an intracellular signaling domain.

In some aspects, the target specific receptor and death domain can be any of those disclosed herein. For example, the target specific receptor can be a MOG specific receptor and the death domain can be a Fas domain.

In some aspects, the antigen binding domain is an antibody fragment or an antigen-binding fragment that specifically binds to an antigen of interest. In some aspects, the antigen binding domain is a LINGO1 domain. In some aspects, the antigen of interest is LINGO1. Thus, in some aspects, the second CAR comprises a LINGO1 antigen binding domain, a transmembrane domain, and an intracellular signaling domain. In some aspects, the antigen targeted by the antigen binding domain can be, but is not limited to, LINGO1, CD19, CD22, CD33, CD70, CD38, EGFR, EPCAM, ERBB2, GPC3, EPHA2, TNFRSF17, SDC1, MS4A1, ROR1, CD133, CD276, TEM1, GD2, CD23, LlCAM, CD174, CD44, SLAMF7, or KDR.

In some aspects, the antigen binding domain, transmembrane domain, and intracellular signaling domain can be any of those disclosed herein.

In some aspects, the second CAR polypeptide further comprises a tag sequence. The tag sequence can be any tag sequence disclosed herein. In some aspects, the tag sequence is located between the antigen binding domain and the transmembrane domain.

In some aspects, the second CAR polypeptide further comprises a hinge region. In some aspects, the hinge region is located between the antigen binding domain and the transmembrane domain. In some aspects, the second CAR comprises a tag sequence and hinge region. In some aspects, the tag sequence can be on the N-terminal or C-terminal end of the hinge region. In some aspects, both the tag sequence and the hinge region can be located between the antigen binding domain and the transmembrane domain.

Disclosed are CAR T cells comprising a first CAR polypeptide comprising a LINGO1 antigen binding domain; and a second CAR polypeptide comprising a MOG specific receptor and a Fas domain.

4. Altered α4β1 Integrin

α4β1 integrin binds to Vascular Cell Adhesion Molecule 1 (VCAM1) present on vascular endothelial cells. This interaction allows for migration of cells comprising a401 integrin to cross the blood brain barrier. Thus, in some aspects, altering the interaction of α4β1 integrin and VCAM1 can reduce the migration of cells across the blood brain barrier.

Disclosed are any of the cells described herein further comprising an altered α4β1 integrin. In some aspects, an altered α4β1 integrin is deleted (in whole or in part) or mutated. In some aspects, the mutated α4β1 integrin is mutated in the first exon. In some aspects, the mutated α4β1 integrin has a frameshift mutation or deletion. In some aspects, a CRISPR/Cas9 approach can be used to remove all, or a portion, of the α4β1 integrin.

In some aspects, the altered α4β1 integrin cannot bind to VCAM1 on vascular endothelial cells.

Disclosed are CAR T cells comprising a CAR polypeptide and a mutated a4b1 integrin, wherein the CAR polypeptide comprises a MOG specific receptor and a Fas domain.

Disclosed are CAR T cells comprising a CAR polypeptide and a mutated a4b1 integrin, wherein the CAR polypeptide comprises a LINGO1 antigen binding domain. The CAR polypeptide can further comprise a transmembrane domain, intracellular domain, hinge domain, and/or or tag sequence as disclosed herein.

Disclosed are CAR T cells comprising a first CAR polypeptide, a second CAR polypeptide, and a mutated a4b1 integrin, wherein the first CAR polypeptide comprises a LINGO1 antigen binding domain, wherein the second CAR polypeptide comprises a MOG specific receptor and a Fas domain. In some aspects, the first CAR polypeptide can further comprise a transmembrane domain, intracellular domain, hinge domain, and/or or tag sequence as disclosed herein.

5. AP-1 Transcription Factors

Some embodiments of the disclosed invention are based on the fact that T cells modified (e.g., genetically) to overexpress and/or contain elevated (e.g., supraphysiologic) levels of one or more AP-1 transcription factors (e.g., c-Jun) display reduced levels of T cell exhaustion (e.g., compared to unmodified T cells expressing normal levels of AP-1 transcription factors). In some aspects, the disclosed CAR T cells can have reduced expansion during production and increasing the levels of one or more AP-1 transcription factors, such as c-Jun, can rescue the reduced expansion.

In some aspects, any of the disclosed CAR T cells can be modified to overexpress and/or contain elevated levels of one or more AP-1 transcription factors. In some aspects, the AP-1 transcription factors are selected from the group consisting of c-Fos, c-Jun, Activating transcription factor (ATF) and Jun dimerization protein (JDP).

In some aspects, the AP-1 transcription factor, such as c-Jun, and the suicide switch CAR or LINGO CAR are expressed from separate expression vector constructs or are co-expressed from a single expression vector construct.

In some aspects, modified to overexpress and/or contain elevated levels of one or more AP-1 transcription factors can include adding in exogenous nucleic acid that encodes for an AP-1 transcription factor, adding in an exogenous AP-1 transcription factor, or providing a composition that activates expression of an endogenous AP-1 transcription factor.

### E. Vectors

Disclosed are vectors comprising the nucleic acid sequence of the disclosed CAR nucleic acid sequences. For example, disclosed are vectors comprising the nucleic acid sequence that encodes one or more of the disclosed CAR polypeptides. In some instances, the vector can be a DNA, a RNA, a plasmid, a cosmid vector, or a viral vector. In some aspects, the vector can be a herpes virus vector, a measles virus vector, a lentivirus vector, an adenoviral vector, and a retrovirus vector. In some instances, the vector can comprise a promoter.

Disclosed are vectors comprising a nucleic acid sequence encoding any of the disclosed CAR polypeptides and a nucleic acid sequence encoding c-Jun.

Disclosed herein are methods of making T cells comprising transducing a T cell with one of the vectors disclosed herein.

### F. Compositions

Disclosed are compositions comprising one or more of the disclosed CAR polypeptides, CAR cells, CAR nucleic acid sequences, or vectors.

1. Pharmaceutical Compositions

In some aspects, the disclosed compositions can be pharmaceutical compositions. Disclosed are compositions comprising one or more of the disclosed CAR polypeptides, CAR cells, CAR nucleic acid sequences, or vectors in combination with a pharmaceutically acceptable carrier. For example, in some aspects, disclosed are pharmaceutical compositions comprising a composition comprising one or more of the CAR T cells disclosed herein and a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, or conjugate of the invention is not compromised. Pharmaceutical compositions may also include one or more active ingredients (in addition to the composition of the invention) such as antimicrobial agents, anti-inflammatory agents, anesthetics, and the like.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the fusion proteins. Thus, compositions can be prepared for parenteral administration that includes fusion proteins dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The pharmaceutical compositions described above can be formulated to include a therapeutically effective amount of a composition disclosed herein. In some aspects, therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to one or more autoimmune diseases or where the patient has a clinically determined predisposition or increased susceptibility (in some cases, a greatly increased susceptibility) to cancer.

The pharmaceutical compositions described herein can be administered to the subject (e.g., a human subject or human patient) in an amount sufficient to delay, reduce, or preferably prevent the onset of clinical disease. Accordingly, in some aspects, the subject is a human subject. In therapeutic applications, compositions are administered to a subject (e.g., a human subject) already with or diagnosed with an autoimmune disease in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effective amount includes amounts that provide a treatment in which the onset or progression of the cancer is delayed, hindered, or prevented, or the autoimmune disease or a symptom of the autoimmune disease is ameliorated. One or more of the symptoms can be less severe. Recovery can be accelerated in an individual who has been treated.

The total effective amount of the conjugates in the pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, 1-2 weeks, or once a month). Alternatively, continuous intravenous infusions sufficient to maintain therapeutically effective concentrations in the blood are also within the scope of the present disclosure.

The pharmaceutical composition may be administered in a number of ways depending on whether local or systemic treatment is desired, and on the area to be treated.

### G. Methods

Any of the disclosed methods can use one or more of the disclosed CAR T cells that comprise an increased amount of c-Jun in order to reduce the chance of T cell exhaustion or decreased expansion.

1. Treating with a LINGO1 Specific CAR T Cell

Disclosed are methods of treating a subject having cancer comprising administering a composition comprising a CAR T cell to a subject having cancer, wherein the CAR T cell comprises a CAR polypeptide comprising a LINGO1 antigen binding domain, a transmembrane domain, and an intracellular signaling domain, wherein the subject having cancer has cancer cells expressing LINGO1, wherein the CAR T cell binds LINGO1 on the cancer cells activating the CAR T cell to kill the cancer cell.

In some aspects, the CAR polypeptide comprising a LINGO1 antigen binding domain, a transmembrane domain, and an intracellular signaling domain can be any of the CAR polypeptides disclosed herein.

2. Treating with a CAR T Cell Having a Suicide Switch

Disclosed are methods of treating cancer comprising administering a composition comprising a CAR T cell to a subject having cancer, wherein the CAR T cell comprises a first CAR polypeptide comprising an over-expressed cancer antigen binding domain, a transmembrane domain, and an intracellular signaling domain; and a second CAR polypeptide comprising a non-cancer specific antigen receptor and a death domain; wherein when the CAR T cell binds a non-cancer specific antigen the second CAR polypeptide activates killing of the CAR T cell. Thus, in some aspects, the second CAR polypeptide is a suicide switch.

In some aspects, the cancer can be any cancer with a known over-expressed cancer antigen. In some aspects, an over-expressed cancer antigen is an antigen present at higher levels on cancer cells compared to non-cancer cells.

In some aspects, the cancer is Ewing's Sarcoma. In some aspects, the over-expressed cancer antigen is an over-expressed Ewing's Sarcoma antigen. In some aspects, the over-expressed Ewing's Sarcoma antigen is LINGO1.

In some aspects, the cancer is neuroblastoma or osteosarcoma. In some aspects, the over-expressed cancer antigen is an over-expressed neuroblastoma or osteosarcoma antigen. In some aspects, the over-expressed neuroblastoma or osteosarcoma antigen is GD2.

In some aspects, the non-cancer specific antigen is a CNS-specific antigen. Thus, in some aspects, the non-cancer specific antigen is MOG.

In some aspects, the CAR T cell further comprises a mutated a4b1 integrin. Therefore, in some aspects, the CAR T cell used for treating a subject can comprise a first CAR polypeptide comprising an over-expressed cancer antigen binding domain, a transmembrane domain, and an intracellular signaling domain; a second CAR polypeptide comprising a non-cancer specific antigen receptor and a death domain; and a mutated a4b1 integrin.

Disclosed are methods of treating Ewing's Sarcoma comprising administering a composition comprising a CAR T cell to a subject having Ewing's Sarcoma, wherein the CAR T cell comprises a first CAR polypeptide comprising a LINGO1 antigen binding domain; and a second CAR polypeptide comprising MOG specific receptor and a Fas domain; wherein upon crossing the blood brain barrier the MOG specific receptor binds MOG on neurons and activates killing of the CAR T cell. Thus, the killing of the CAR T cell is a cell suicide (i.e. apoptosis) caused by the suicide switch. In some aspects, the CAR T cell further comprises a mutated a4b1 integrin.

Also disclosed are methods of treating a subject having self-reactive T cells comprising administering a composition comprising a CAR T cell to a subject having self-reactive T cells, wherein the CAR T cell comprises a suicide switch. In some aspects, a subjects T cells can be removed, genetically modified to comprise a CAR comprising a suicide switch thereby generating a genetically modified T cell, then administering the genetically modified T cells back to the subject. In some aspects, the genetically modified T cells comprise a CAR comprising a target specific receptor and a death domain, wherein the target specific receptor is an autoantigen specific receptor. In some aspects, the T cells removed from the subject would be tested to determine the autoantigen the T cells are specific to. A CAR having a target specific receptor directed to the same autoantigen can then be introduced into the T cell. Once the genetically modified T cell has been administered back to the subject, the CAR will bind the same autoantigen the natural T cell receptor would bind thus triggering cell suicide. This provides a mechanism for killing T cells that target self proteins (autoantigens).

In some aspects, the subject having self-reactive T cells has an autoimmune disease.

3. Reducing Migration Across BBB

Disclosed are methods of reducing migration of cells across the blood brain barrier comprising administering a composition comprising a cell to a subject wherein the cell comprises an altered α4β1 integrin. In some aspects, the altered α4β1 integrin can be any of the altered α4β1 integrins described herein.

In some aspects, the cell is a T cell. In some aspects, the T cell is a CAR T cell.

In some aspects, the CAR T cell comprises a mutated α4β1 integrin and a CAR comprising a target specific receptor and a death domain. In some aspects, the CAR comprises a MOG specific receptor and a Fas domain.

4. Inducing Apoptosis

Disclosed are methods of inducing apoptosis of a CAR T cell comprising administering a composition comprising a CAR T cell to a subject wherein the CAR T cell comprises a CAR polypeptide comprising a target specific receptor and a death domain wherein when the target specific antigen receptor of the CAR T cell binds the target, the death domain activates killing of the CAR T cell. In some aspects, the target of the target specific antigen receptor is present on healthy cells only.

Disclosed are methods of inducing apoptosis of a CAR T cell comprising administering a composition comprising a CAR T cell to a subject wherein the CAR T cell comprises a CAR polypeptide comprising a healthy cell specific antigen receptor and a death domain, wherein when the healthy cell specific antigen receptor of the CAR T cell binds a healthy cell specific antigen on a healthy cell, the death domain activates killing of the CAR T cell.

In some aspects, a healthy cell specific antigen receptor is a MOG specific receptor. In some aspects, the CAR polypeptide can be any of the suicide switches described herein.

In some aspects, the CAR T cell further comprises a second CAR polypeptide. In some aspects, the second CAR polypeptide can be any of the CAR polypeptides described herein comprising an antigen binding domain, a transmembrane domain, and an intracellular signaling domain. In particular, in some aspects, the second CAR polypeptide can be any of the LINGO1 specific CAR polypeptides described herein.

In some aspects, the CAR T cell can further comprise an altered α4β1 integrin. In some aspects, the altered α4β1 integrin can be any of the altered α4β1 integrins described herein.

### H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the disclosed CAR polypeptides, altered α4β1 integrins, and/or CAR cells. Also disclosed are kits comprising one or more of the nucleic acid sequences that encode the disclosed CAR polypeptides and/or altered α4β1 integrins.

### EXAMPLES

### A. Example 1

Ewing Sarcoma is a solid tumor that is thought to arise from mesenchymal stem cells in pediatric patients. Currently, only 20% of patients with metastatic/recurrent Ewing sarcoma can be cured by currently available treatments.

Chimeric antigen receptor (CAR) T cells are gaining popularity as therapeutic options in hematologic and solid tumor malignancies. This approach relies on the recognition of surface antigens on tumor cells by CAR-transgenic T cells (Almasbak et al. J Immunol Res. 2016; 2016:5474602). However, there are currently no CAR T cell approaches available for the treatment of Ewing sarcoma due to the lack of appropriate target antigens.

It has previously been demonstrated that there is surface expression of Leucine-Rich Repeat and Ig Domain Protein 1 (LINGO1) on Ewing sarcoma cells and have shown efficient tumor cell killing with a monoclonal antibody in vitro (Town J, Pais et al. Proc Natl Acad Sci USA. 2016; 113(13):3603-8). However, LINGO1 is also expressed on healthy neurons (Mi et al. Nat Neurosci. 2004; 7(3):221-8), currently prohibiting its clinical use due to potential on-target off-tumor toxicities. Described herein are LINGO1 CAR T cells and two safety mechanisms to prevent targeting of the central nervous system (CNS).

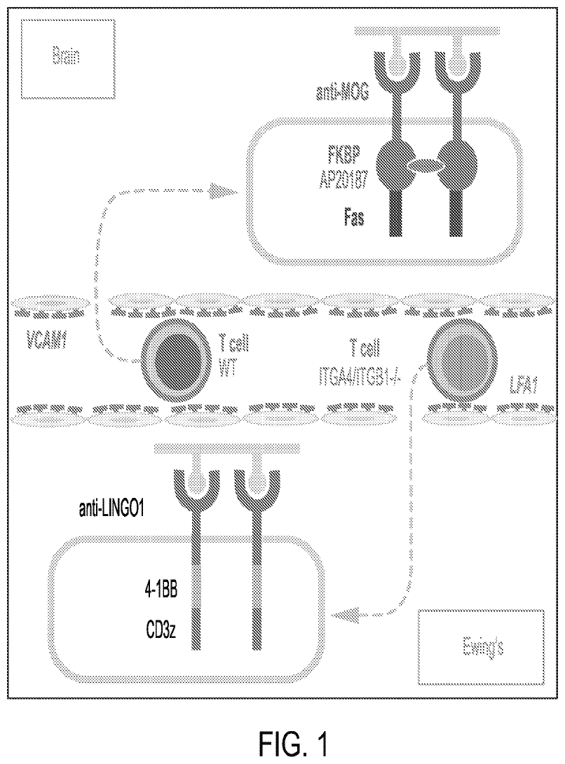

The first safety mechanism can prevent CAR T cells from crossing the blood-brain barrier. Using a CRISPR/Cas9-based approach, there is a disruption of the binding of α4β1 integrin (ITGA4/ITGB1) expressed on T cells to Vascular Cell Adhesion Molecule 1 (VCAM1), the main adhesion molecule involved in blood-brain barrier migration, which is present on vascular endothelial cells (Takeshita et al. Immunol Rev. 2012; 248(1):228-39). Second, in order to eliminate CAR T cells localizing to the CNS, a CNS-sensing suicide switch, which rapidly induces apoptosis in T cells when encountering the CNS-specific surface antigen Myelin Oligodendrocyte Glycoprotein (MOG) (Annenkov et al. Biochim Biophys Acta. 2011; 1813(8):1428-37), can be used (FIG. 1).

Described herein is a cellular immunotherapy for Ewing sarcoma and two safety mechanisms to shape CAR T cell selectivity through alteration of a specific adhesion event and a novel molecularly targeted suicide switch.

1. Significance

The successful knockout of ITGA4/ITGB1 in Jurkat-Luc cells using CRISPR/Cas9 approach has been performed. This knockout disrupts binding to VCAM-1 and dramatically reduces T cell adhesion. Significantly limiting T cell migration across the blood brain barrier in vitro and in vivo is described herein.

The disclosed LINGO-1 CAR T cells with its safety mechanism can enhance their selectivity. The current experiments can 1) demonstrate that LINGO1 CAR T cells are effective against Ewing sarcoma and 2) that the two safety mechanisms described in this proposal are feasible and able to shape CAR T cell selectivity through alteration of specific adhesion events and a novel molecularly targeted suicide switch. These approaches can then have a substantial impact on future adoptive T cell therapies, in particular when targeting solid tumor malignancies which don't always have a unique or specific target antigen and help to overcome these barriers.

This approach can be used to overcome a central barrier to the development of CAR T cells for the treatment of pediatric cancers. Specifically, an approach to allow the targeting of a tumor antigen which shows expression in the CNS by combining a tissue-sensing suicide switch was developed, the first CAR T cells targeting LINGO1, as well as the first CRISPR/Cas9-based approach to prevent migration of CAR T cells across the blood-brain barrier.

2. Background

Ewing Sarcoma is a solid tumor that is thought to arise from mesenchymal stem cells in pediatric and young adult patients. Treatment for localized Ewing Sarcoma consists of multi-agent neoadjuvant chemotherapy in compressed cycles. This is followed by local control which includes surgery and/or radiation followed by adjuvant chemotherapy. This therapy comes with significant side effects including risks for secondary malignancies, cardiotoxicity and infertility. This confers survival rates of approximately 70%, (Grier et al. N Engl J Med. 2003; 348(8):694-701), however in relapsed patients or patients with upfront metastatic disease, overall survival is less than 20% (Subbiah et al. Curr Treat Options Oncol. 2009; 10(1-2):126-40). Current immunotherapy options are limited and still in clinical trials. A randomized phase III trial lead by the Children's Oncology group (COG) studied the use of ganitumab, a monoclonal antibody against Insulin Growth Factor Receptor 1 (IGF1R) in upfront therapy for metastatic Ewing Sarcoma along with conventional chemotherapy (Rizk et al. Pharmgenomics Pers Med. 2019; 12:9-14). However the study was closed to accrual in the spring of 2019 and ganitumab was discontinued based upon a lack of significant benefit of the addition of ganitumab, as well as the potential for increased toxicity, such as pneumonitis. Furthermore, a clinical trial testing checkpoint inhibitor pembrolizumab in adults with Ewing Sarcoma demonstrated a lack of objective responses. This was attributed to a low mutational burden and lack of PD-L1 expression in Ewing Sarcoma tumors (Tawbi et al. Lancet Oncol. 2017; 18(11):1493-501).

Other immune-based therapy options are being explored in adults with Ewing Sarcoma, such as vaccination approaches, which are currently in clinical trials (Ghisoli et al. Mol Ther. 2016; 24(8):1478-83). While cellular immunotherapies using engineered T cells have shown impressive clinical activity in pediatric B cell malignancies, so far it has proven difficult to translate these successes to other types of cancer (Maude et al. N Engl J Med. 2014; 371(16):1507-17). These approaches are generally based on the introduction of receptors into the patient's own T cells to redirect them towards tumor cells, with chimeric antigen receptors (CARs) targeting tumor-specific surface antigens. CAR T cells targeting IGF1R and tyrosine kinase-like orphan receptor 1 (ROR1) have been explored in the preclinical setting in Ewing Sarcoma, however these have not yet made it into the clinic and a key problem in the development of effective CAR T cell therapies remains the expression of potential tumor antigens on healthy tissues leading to on-target off-tumor toxicities. To date, no effective immunotherapy has been incorporated into Ewing Sarcoma therapy.

The expression of surface antigen LINGO1 in Ewing sarcoma has been studied. LINGO1 was expressed in over 90% of Ewing Sarcoma tumors and treatment with an antibody-drug conjugate targeting LINGO1 resulted in the efficient killing of Ewing sarcoma cells in vitro (Town J, Pais et al. Proc Natl Acad Sci USA. 2016; 113(13):3603-8). However, while otherwise showing a highly restricted expression in healthy tissues, LINGO1 could also be detected in the central nervous system (CNS), currently prohibiting its use as a therapeutic target.

The disclosed experiments are designed for the selectivity of LINGO1 CAR T cells by preventing CNS targeting through the addition of two safety mechanisms. LINGO1-specific CAR T cells equipped with two safety mechanisms to prevent CNS targeting by 1) reducing migration across the blood-brain barrier (BBB) and 2) eliminating T cells after entering the CNS can be created (FIG. 1). To achieve this a CRISPR/Cas9-based approach was developed to disrupt the binding of activated α4β1 integrin expressed on T cells to Vascular Cell Adhesion Molecule 1 (VCAM1) a key adhesion molecule on vascular endothelial cells involved in blood-brain barrier migration (Takeshita et al. Immunol Rev. 2012; 248(1):228-39) Second, in order to eliminate CAR T cells in the CNS, a tissue-sensing miFas suicide switch was developed that induces apoptosis in T cells when encountering the CNS-specific antigen Myelin Oligodendrocyte Glycoprotein (MOG), which is absent from any other somatic tissues as well as Ewing sarcoma cells. In addition, this receptor is equipped with a small molecule-inducible FKBP homodimerization domain (Spencer et al. 1993; 262(5136):1019-24).

3. Results

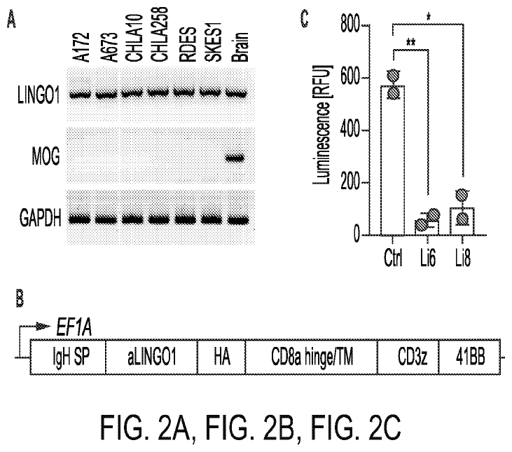

The main goal of this project is to develop the first CAR T cell approach targeting LINGO1 on Ewing Sarcoma (EWS) cells and to develop two approaches to shape the selectivity of CAR T cells for tumor cells. The presence of LINGO on Ewing sarcoma cell lines and in human brain mRNA has been confirmed as well as the lack of MOG on Ewing sarcoma and its presence in Brain mRNA via reverse transcription (RT)-PCR (FIG. 2A). In addition, MOG expression has been studied extensively by other groups (Solly et al. Glia. 1996; 18(1):39-48); (Brunner et al. J Neurochem. 1989; 52(1):296-304); (Bruno et al. Eur J Immunol. 2002; 32(10):2737-47). LINGO1-specific CAR constructs (FIG. 2B) were generated based on previously described anti-LINGO1 antibody sequences and primary human LINGO1 CAR T cells were generated (Town J, Pais et al. Proc Natl Acad Sci USA. 2016; 113(13):3603-8). LINGO1 CAR T cells but not GFP-transduced T cells efficiently killed K562 cells transduced with LINGO1 (FIG. 2C).

To test the specificity of the constructs, K562 cells expressing MOG, LINGO1, or both antigens together were also generated.

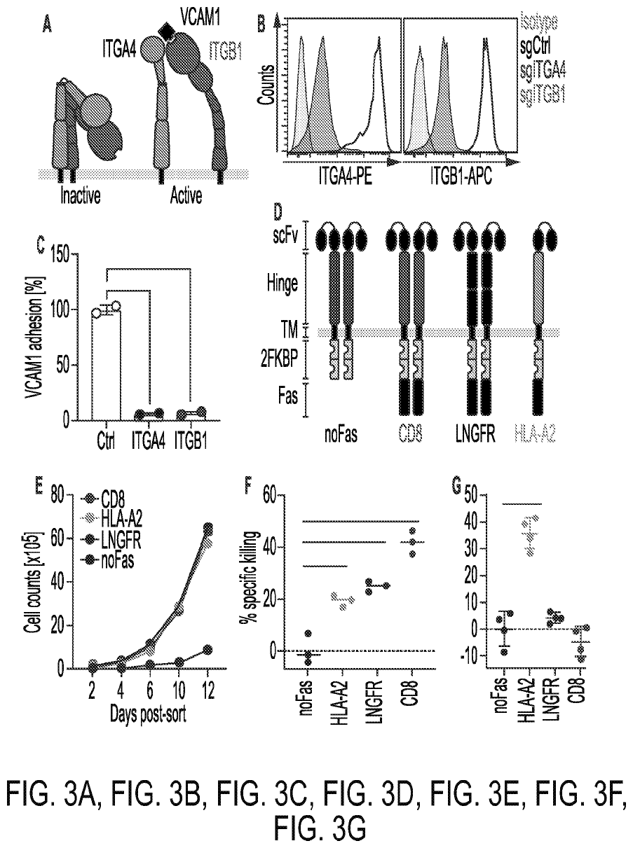

In order to limit the migration of LINGO1 CAR T cells across the blood brain barrier, we next performed a CRISPR/Cas9 knockout of the individual components of the α4β1 integrin heterodimer VLA4: α4 (ITGA4) and β1 (ITGB1). Jurkat T cells express high levels of ITGA4 and ITGB1 (FIG. 3A) and the α4β1 heterodimer is constitutively active in these cells (Yednock et al. J Biol Chem. 1995; 270(48):28740-50). Comparing multiple CRISPR guide RNA sequences, constructs were identified which result in the efficient targeting of either gene. Treatment of Jurkat cells with these ITGA4- or ITGB1-specific single guide RNA constructs and Cas9 protein resulted in the efficient knockout and loss of both subunits from the cell surface (FIG. 3B).

A luminescence-based T cell adhesion assay was next generated by generating Jurkat T cells stably expressing firefly luciferase. Knocking out ITGA4 or ITGB1 in these cells led to dramatically reduced adhesion to immobilized recombinant human VCAM1 (FIG. 3C). For subsequent assays the ITGA4 knockout was used, as ITGA4 shows a more limited use in other integrin heterodimers (Zent et al. New York: Springer; 2010. xii, 314 p. p).

As a second safety mechanism to prevent the targeting of normal brain cells expressing LINGO1 by CAR T cells, tissue-sensing membrane-bound inducible Fas (miFas) receptors (FIG. 3D) were also generated, to be co-expressed by LINGO1 CAR T cells (FIG. 1). These receptors combine a scFv domain targeting MOG or CD19 with an intracellular Fas domain. MOG is expressed throughout the CNS (FIG. 2A) and the MOG-specific scFv has been shown to be cross-reactive to human and mouse MOG (Breij et al. J Neuroimmunol. 2006; 176(1-2):106-14); (Morris-Downes et al. J Neuroimmunol. 2002; 125(1-2):114-24). After cross-linking, Fas has been shown to rapidly induce killing of transgenic T cells (Straathof et al. Blood. 2005; 105(11):4247-54). A variety of miFas receptors utilizing different hinge domains was generated to facilitate Fas assembly and strong surface expression of all variants in Jurkat cells was and efficient expansion of primary human T cells expressing the same constructs was observed (FIG. 3E). Interestingly, both monomeric and dimeric miFas constructs expanded well, while the previously described LNGFR-based construct (Straathof et al. Blood. 2005; 105(11):4247-54) showed only relatively modest growth. Dying of primary human T cells expressing miFas constructs were generated after addition of the chemical inducer of FKBP dimerization AP20187. Efficient induction of cell death for all three constructs was observed (FIG. 3F). However, when analyzing whether the constructs were able to induce cell death in T cell line J76 through recognition of a tissue-specific surface antigen, only the HLA-A2-based construct induced cell death when co-cultured with CD19-transgenic K562 cells was found (FIG. 3G). Therefore, the HLA-A2-based construct was selected as the lead candidate.

4. Design and Methods

i. Determine Cytotoxic Activity of Genetically Engineered LINGO1 CAR T Cells Against Ewing Sarcoma.

a. Determine Cytotoxic Activity of LINGO1 CAR T Cells Against Ewing Sarcoma Cell Lines.

Killing of K562 cells and K562 cells expressing LINGO1 can be determined, as well as six Ewing sarcoma cell lines expressing LINGO1 (FIG. 2A) transduced with luciferase by LINGO1 CAR T cells using the luminescence-based cytotoxicity assay described above. IFNG and IL2 levels can also be determined in co-culture supernatants by ELISA. LINGO1 CAR T cells are then co-cultured with healthy neurons and their targeting determined using a flow cytometry based cytotoxicity assay.

b. Determine In Vivo Activity of LINGO1 CAR T Cells Against Ewing Sarcoma.

To determine whether LINGO1 CAR T cells are also effective against larger established Ewing sarcoma tumors in vivo, a xenograft model using the NOD.Cg-Prkdc^{scid}Il2rg^{tm1Wjl}/SzJ (NSG) strain of mice is used. Human Ewing sarcoma cell lines expressing luciferase will be injected subcutaneously and once tumors reach a volume of 100-200 mm3, LINGO1 or control CAR T cells can be injected via tail vein. Tumor burden can be monitored weekly using in vivo bioluminescence imaging. Survival can be analyzed by log-rank test and Kaplan-Meier curves.

c. Results

Efficient killing of K562 cells transduced with a LINGO1 expression construct was observed by the LINGO1 CAR T cells. Similar levels of killing of Ewing sarcoma cells can be seen, which express high levels of LINGO1 mRNA and were efficiently targeted by a monoclonal antibody, by LINGO1 CAR T cells. However, in case low levels of Ewing sarcoma cell killing is observed, the levels of CAR T cell activation and generation of perform and granzyme B can be characterized in co-cultures with Ewing sarcoma cells. The use of alternative LINGO1-specific antibodies for the engineering of CAR constructs can also be examined.

ii. Validate CNS-Sensing Suicide Switch and CRISPR/Cas9-Based Safety Mechanism to Prevent CAR T Cell Migration Across the Blood-Brain Barrier.

a. Determine Effect of α4β1 Integrin Knockout on Blood Brain Barrier Migration of CAR T Cells.

Jurkat cells and primary human T cells transduced with Cas9 and negative control or ITGA4-specific guide RNAs are produced. In a transwell assay, migration of Jurkat cells across a layer of human endothelial HUVEC cells before and after treatment with TNFα can be determined to induce expression of VCAM1 (Kim et al. Exp Mol Med. 2017; 49(2):e294). The migration of Jurkat cells across the BBB (10 animals=5 animals per sgControl or sgITGA4) is quantified in an established mouse model, which showed that migration of Jurkat cells can be inhibited with an ITGA4-specific monoclonal antibody (Coisne et al. J Immunol. 2009; 182(10):5909-13). Migration of primary T cells can be determined in the same in vivo model. T cells can undergo ITGA4 or control knockout and can be either left untreated or treated with recombinant SDF1 or CD3/CD28 beads to induce the active conformation of a401 integrin. T cells can be injected via tail vein (30 animals=5 animals per knockout condition/treatment) and animals sacrificed 2 days after T cell injection. Single cell suspensions of brains can be analyzed by flow cytometry for human T cell frequencies and activation.

b. Determine Kinetics of miFas-Induced CAR T Cell Apoptosis.

Primary human T cells can be generated expressing miFas variants expressing the HLA-A2 extracellular domain with and without MOG specific scFv as this miFas variant has demonstrated the most potent and specific induction of apoptosis in j76-Tcells. Surface expression of the different receptors can be confirmed by flow cytometry after staining with a myc-specific antibody. Induction of killing of miFas-expressing T cells can be determined in the presence of MOG+ K562 cells, MOG− K562 cells as a negative control, or through FKBP-mediated chemically induced cross-linking using AP20187. Apoptosis of T cells can be measured by flow cytometry after staining with an anti-CD3 antibody and using counting beads to determine the viability of miFas expressing primary T cells at different timepoints (30 min, 1 h, 2 h, 4 h, 8 h, 16 h, 24 h).

c. Characterize Selectivity of LINGO1 CAR/ITGA4 Knockout/MOG-miFas T Cells for Ewing Sarcoma In Vivo.

To evaluate the combinatorial approach, an in vivo xenograft model of Ewing sarcoma can be established using NSG mice. Ewing sarcoma cell line A673 can be injected subcutaneously in Matrigel and tumors can be allowed to grow until they reach a volume of 100-200 mm3. Primary human T cells expressing wild-type α4β1 integrin or ITGA4 knockout cells, expressing a LINGO1-specific CAR or a negative control CAR, as well as the MOG-miFas receptor or a negative control miFas receptor can be injected by tail vein (40 animals=5 animals per integrin condition/CAR condition/miFas condition). Tumor burden and survival can be determined and mouse brains and tumors analyzed for T cell frequencies, activation, and ITGA4 expression.

d. Results

T cells are able to express high levels of miFas receptors and the CD19 and MOG-specific scFv clones as well as the intracellular Fas domain have previously been used for different types of chimeric receptors. The tandem FKBP domains required for chemical induction of dimerization can be omitted as well as replacing hinge and transmembrane domains with the respective domains of low-affinity nerve growth factor receptor (LNGFR), which have been shown to be able to facilitate antibody-mediated induction of intracellular Fas signaling (Straathof et al. Blood. 2005; 105(11):4247-54) (FIG. 3A). Other MOG-specific scFv sequences which have been described before can be used or new, fully human MOG-specific antibodies can be developed using a naïve antibody phage display library containing >10¹⁰ scFv clones.

### B. Example 2

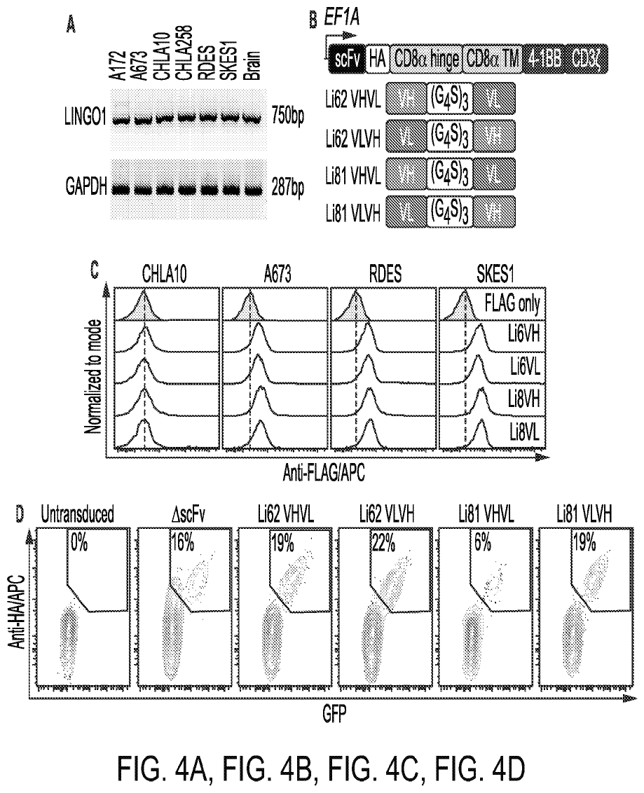

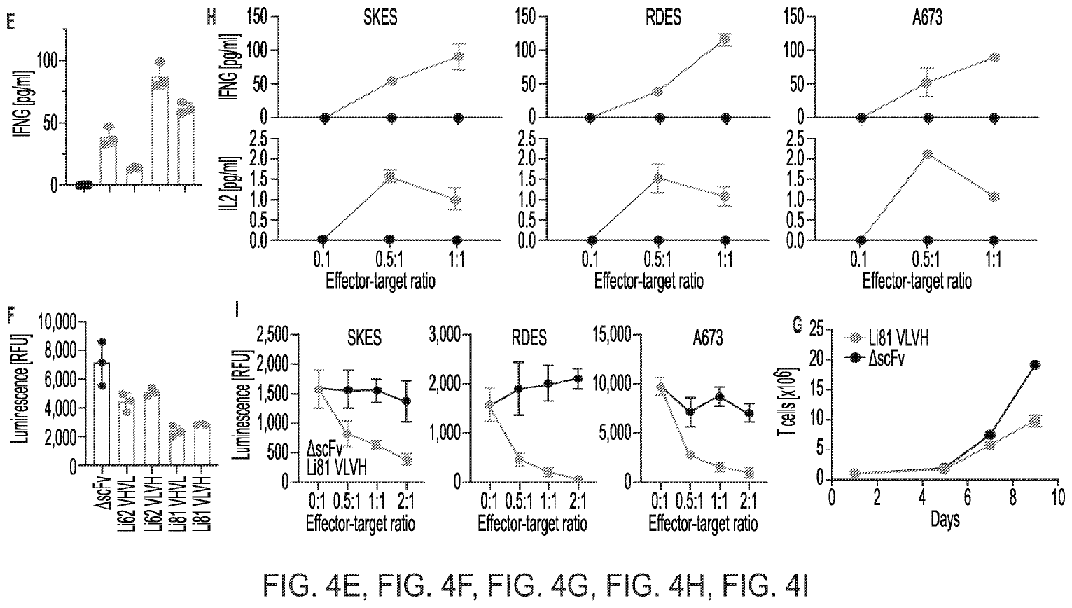

Ewing sarcoma is a malignant solid tumor affecting approximately 200 pediatric and young adult patients in the U.S. per year. Survival of patients with relapsed disease or upfront metastatic disease is less than 20% (Casey et al. Front Oncol 9, 537 (2019) demonstrating the need for new therapeutic approaches. Only few surface antigens specific for Ewing sarcoma have been identified so far (Town et al. Proc Natl Acad Sci USA 113, 3603-3608 (2016). One of these antigens, LINGO1, a component of the Nogo receptor, which is widely expressed in the central nervous system (CNS) (Zhang et al. J Biol Chem 288, 12152-12160 (2013), has been shown to be commonly expressed on Ewing sarcoma cells (Town et al. Proc Natl Acad Sci USA 113, 3603-3608 (2016) (FIG. 4A) and an antibody-drug conjugate targeting LINGO1 showed efficacy against Ewing sarcoma in vitro (Town et al. Proc Natl Acad Sci USA 113, 3603-3608 (2016). While trafficking of this antibody to the brain may effectively be limited by the BBB (Town et al. Proc Natl Acad Sci USA 113, 3603-3608 (2016), activated T cells as well as CAR T cells have been shown to be able to readily cross the BBB (Engelhardt et al. Nat Immunol 18, 123-131 (2017). Transendothelial migration of T cells across the BBB relies on high-affinity binding by T cells via the integrin α4β1 heterodimer to VCAM1 expressed on endothelial cells (Takeshita et al. Immunol Rev 248, 228-239 (2012). The monoclonal integrin α4 antibody natalizumab blocking this interaction has been shown to dramatically reduce migration of T cells to the CNS in patients with multiple sclerosis (Coisne et al. J Immunol 182, 5909-5913 (2009) without increasing the incidence of cancer in these patients (Alping et al. Ann Neurol 87, 688-699 (2020) indicating that this interaction predominantly affects T cell migration across the BBB (Coisne et al. J Immunol 182, 5909-5913 (2009). Preventing T cell migration across the BBB by disrupting the ITGA4/VCAM1 interaction may allow the targeting of antigens like LINGO1, which show shared expression on tumor cells and in the CNS (Zhang et al. J Biol Chem 288, 12152-12160 (2013).

CAR T cells targeting LINGO1 for the treatment of Ewing sarcoma were developed. Two previously described IgG antibody clones (Town et al. Proc Natl Acad Sci USA 113, 3603-3608 (2016) against LINGO1 were reformatted into single-chain Fv (scFv) domains either in VL-VH or VH-VL orientation (FIG. 4B) and CAR constructs were generated based on these scFvs. All 4 scFvs stained Ewing sarcoma cell lines (FIG. 4C) and all CARs based on these antibodies showed strong surface expression in primary human T cells (FIG. 4D). In co-cultures with Ewing sarcoma cell line A673, all CAR T cells showed IFNγ secretion (FIG. 4E) as well as killing of the tumor cells compared to negative control ΔscFv T cells (FIG. 4F). Based on these results, Li81 VLVH was pursued as the lead candidate for the following experiments. LINGO CAR T cells based on this construct showed slightly reduced expansion compared to ΔscFv CAR T cells (FIG. 4G) but dose-dependent production of effector cytokines (FIG. 4H) and strong anti-tumor activity against different Ewing sarcoma cell lines in vitro (FIG. 4I) and in vivo (FIG. 4J).

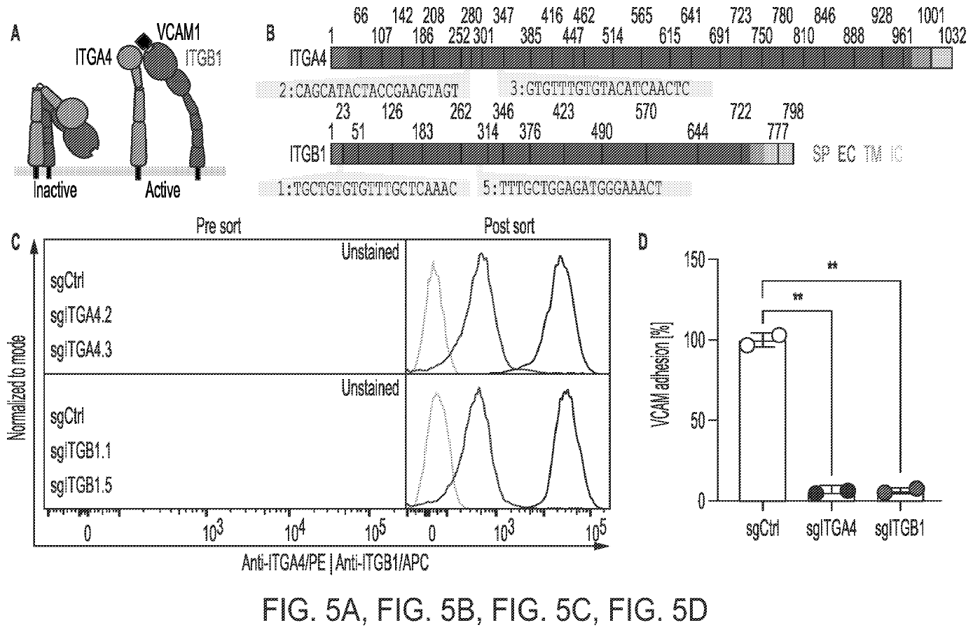

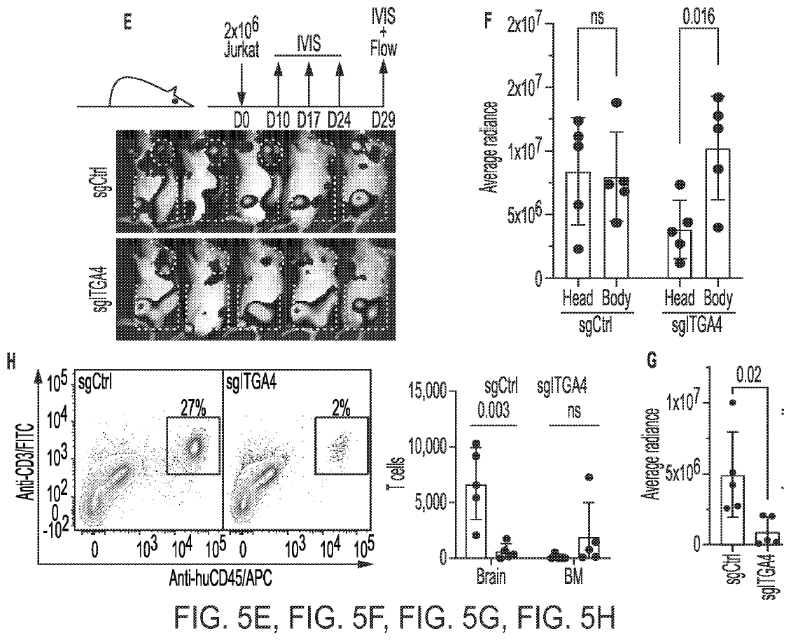

Next, a genetic approach was developed to disrupt binding of active integrin a4b1 expressed on CAR T cells to VCAM1 required for transendothelial BBB migration (Takeshita et al. Immunol Rev 248, 228-239 (2012) (FIG. 5A). CRISPR guide RNA (gRNA) constructs targeting exons coding for the signal peptides or extracellular domains of ITGA4 or ITGB1 were generated (FIG. 5B). After transducing Jurkat cells expressing constitutively active integrin a4b1 with gRNA constructs and Cas9, the most efficient downregulation using sgITGA4 gRNA3 (FIG. 5C) and significantly reduced binding of T cells to recombinant VCAM1 was observed (FIG. 5D). Based on these findings, as well as the more limited use of ITGA4 in other integrin heterodimers potentially affecting other binding events, and the proven safety of targeting ITGA4 by natalizumab, this construct was selected for further development. It has previously been shown that the structural requirements of human and mouse integrin a4b1 binding to mouse VCAM1 are the same (Coisne et al. J Immunol 182, 5909-5913 (2009) and whether ITGA4 knockout is able to limit Jurkat localization to the CNS was determined in a mouse model (FIG. 5E). While mice injected with wildtype Jurkat cells showed comparable luminescence in the head and body, it was observed that the signal was significantly lower in the head of animals injected with Jurkat cells after ITGA4 knockout (FIG. 5F). Indeed, analyzing the explanted brains, total IVIS signal (FIG. 5G) and total human T cell numbers (FIG. 5H) were significantly lower in ITGA4^{ko} animals.

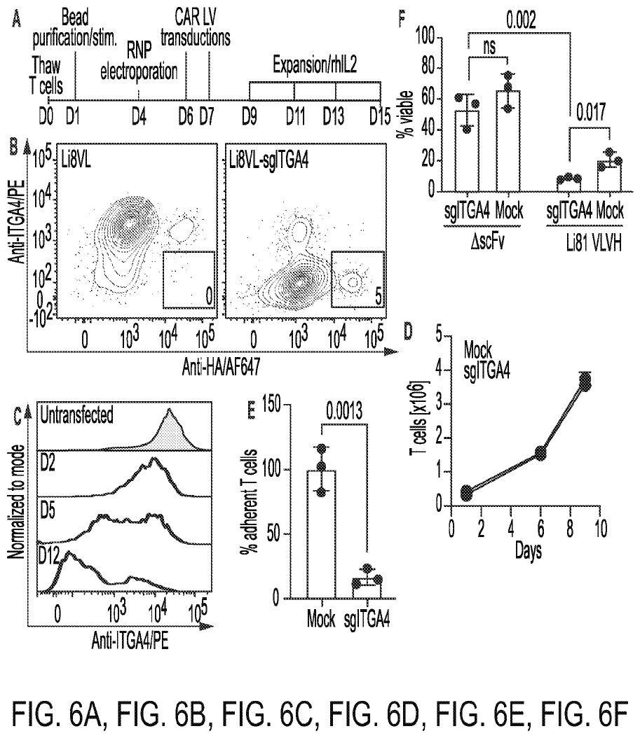

Efficient T cell migration is required for the targeting of solid tumor cells and it has been shown that T cell recruitment to tumor sites may predominantly rely on the LFA/ICAM1 axis. To determine the effects of ITGA4ko on tumor localization and CAR T cell function, primary human ITGA4^{ko} LINGO1 CAR T cells were generated (FIG. 6A) and cells with both the ITGA4 knockout as well as LINGO1 CAR surface expression were generated (FIG. 6B). Loss of ITGA4 from the T cell surface was achieved within 12 days (FIG. 6C) and resulted in significantly reduced adhesion of primary T cells to recombinant VCAM1 (FIG. 6D). Interestingly, killing of Ewing sarcoma cells by LINGO1 CAR T cells was significantly enhanced by ITGA4 knockout in vitro (FIG. 6E) and in vivo (FIG. 6F). In addition, substantially reduced CAR T cell numbers were observed in the brains of animals injected with T cells carrying the ITGA4 knockout.

### C. Example 3

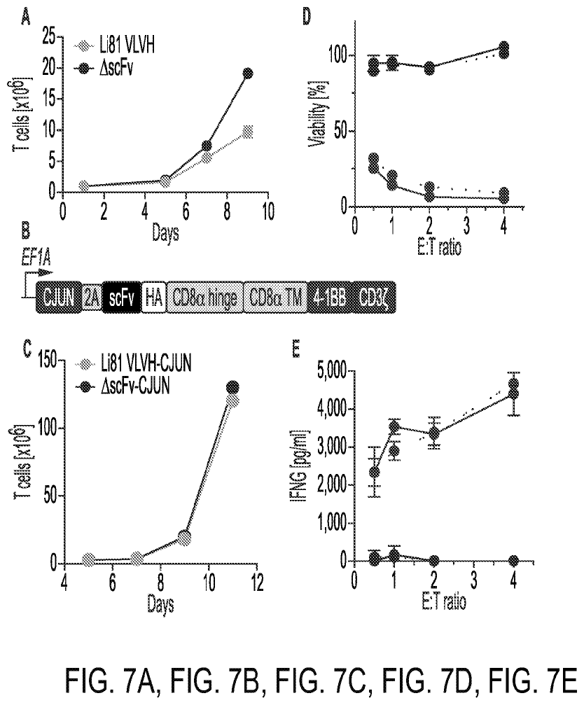

Reduced expansion of LINGO1 CAR T cells can be rescued by CJUN overexpression while maintaining anti-tumor activity (see FIG. 7). FIG. 7A shows that CAR T cells targeting LINGO1 using the Li81 scFv have reduced expansion during CAR T cell production compared to T cells expressing a CAR without a binding domain. This may be a sign of tonic signaling, an antigen-independent stimulation of the T cells via spontaneous aggregation of CARs on the T cell surface (Long et al. Nat Med 21, 581-590, 2015). Overexpression of c-Jun has been suggested to minimize tonic signaling effects on CAR T cells (Lynn et al. Nature 576, 293-300, 2019). FIG. 7C shows that expressing the CAR together with the full-length sequence of human CJUN rescues expansion as shown by almost identical T cell numbers after a 9-day CAR T cell production protocol. FIGS. 7D and 7E show that LINGO1 CAR T cells expressing c-Jun maintain their anti-tumor activity as well as effector cytokine expression in an overnight co-culture cytotoxicity assay targeting the Ewing sarcoma cell line A673 with or without ITGA4 knockout at different effector-target ratios. c-Jun overexpression can enhance the clinical anti-tumor activity of LINGO1 CAR T cells.

Those skilled in the art will recognize, or be able to ascertain using no more than routine experimentation, many equivalents to the specific embodiments of the method and compositions described herein. Such equivalents are intended to be encompassed by the following claims.

We claim:

1. A chimeric antigen receptor (CAR) comprising a target specific receptor and a death domain.

2. The CAR polypeptide of claim 1, wherein the target specific receptor is a myelin oligodendrocyte glycoprotein (MOG) specific receptor.

3. The CAR polypeptide of any of claims 1-2, wherein the target specific receptor is an antibody or antigen binding fragment thereof.

4. The CAR polypeptide of claim 3, wherein the antigen binding fragment is a single chain variable fragment (scFv) domain.

5. The CAR polypeptide of claim 2, wherein the MOG specific receptor is a MOG specific scFv.

6. The CAR polypeptide of any of claims 1-5, wherein the death domain is a Fas domain.

7. The CAR polypeptide of any of claims 1-6, wherein the target specific receptor and the death domain are conjugated to each other.

8. The CAR polypeptide of any of claims 1-7, wherein the target specific receptor and the death domain form a fusion protein.

9. The CAR polypeptide of any of claims 1-8, further comprising a hinge region between the target specific receptor and the death domain.

10. The CAR polypeptide of claim 9, wherein the hinge region is monomeric.

11. The CAR polypeptide of claim 10, wherein the monomeric hinge region is HLA-A2.

12. A CAR polypeptide comprising a Leucine-Rich Repeat and Ig Domain Protein 1 (LINGO1) antigen binding domain, a transmembrane domain, and an intracellular signaling domain.

13. The CAR polypeptide of claim 12, wherein the transmembrane domain comprises a CD8α domain, CD3ζ, FcεR1γ, CD4, CD7, CD28, OX40, or H2-Kb.

14. The CAR polypeptide of any one of claims 12-13, wherein the transmembrane domain is located between the antigen binding domain and the intracellular signaling domain.

15. The CAR polypeptide of claims 12-14, wherein the intracellular signaling domain comprises a co-stimulatory signaling region.

16. The CAR polypeptide of claim 15, wherein the co-stimulatory signaling region comprises the cytoplasmic domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

17. The CAR polypeptide of any one of claims 13-16, wherein the intracellular signaling domain is a T cell signaling domain.

18. The CAR polypeptide of any one of claims 13-17, wherein the intracellular signaling domain comprises a CD3 zeta (CD3ζ) signaling domain.

19. The CAR polypeptide of any one of claims 13-18, wherein the intracellular signaling domain comprises a CD3ζ signaling domain and a co-stimulatory signaling region, wherein the co-stimulatory signaling region comprises the cytoplasmic domain of CD28 or 4-1BB.

20. The CAR polypeptide of any one of claims 13-19, further comprising a tag sequence.

21. The CAR polypeptide of claim 20, wherein the tag sequence is located between the antigen binding domain and the transmembrane domain.

22. The CAR polypeptide of any one of claims 20-21 wherein the tag sequence is a hemagglutinin tag.

23. The CAR polypeptide of any one of claims 13-22, further comprising a hinge region.

24. The CAR polypeptide of claim 23, wherein the hinge region is located between the antigen binding domain and the transmembrane domain.

25. A cell comprising the CAR polypeptide of claims 1-11.

26. The cell of claim 25, wherein the cell is a T cell.

27. A cell comprising the CAR polypeptide of claims 12-24.

28. The cell of claim 27, wherein the cell is a T cell.

29. The cell of any one of claims 25-26, further comprising a second CAR polypeptide, wherein the second CAR polypeptide comprises an antigen binding domain, a transmembrane domain, and an intracellular signaling domain.

30. The cell of claim 29, wherein the antigen binding domain is an antibody fragment or an antigen-binding fragment that specifically binds to an antigen of interest.

31. The cell of any one of claims 29-30, wherein the antigen binding domain is a LINGO1 domain.

32. The cell of any one of claims 30-31, wherein the antigen of interest is LINGO1.

33. The cell of any one of claims 29-32, wherein the transmembrane domain comprises a CD8α domain, CD3ζ, FcεR1γ, CD4, CD7, CD28, OX40, or H2-Kb.

34. The cell of any one of claims 29-33, wherein the transmembrane domain is located between the antigen binding domain and the intracellular signaling domain.

35. The cell of any one of claims 29-34, wherein the intracellular signaling domain comprises a co-stimulatory signaling region.

36. The cell of claim 35, wherein the co-stimulatory signaling region comprises the cytoplasmic domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

37. The cell of any one of claims 29-36, wherein the intracellular signaling domain is a T cell signaling domain.

38. The cell of any one of claims 29-37, wherein the intracellular signaling domain comprises a CD3 zeta (CD3ζ) signaling domain.

39. The cell of any one of claims 29-38, wherein the intracellular signaling domain comprises a CD3ζ signaling domain and a co-stimulatory signaling region, wherein the co-stimulatory signaling region comprises the cytoplasmic domain of CD28 or 4-1BB.

40. The cell of any one of claims 29-39, wherein the second CAR polypeptide further comprises a tag sequence.

41. The cell of claim 40, wherein the tag sequence is located between the antigen binding domain and the transmembrane domain.

42. The cell of any one of claims 40-41, wherein the tag sequence is a hemagglutinin tag.

43. The cell of any one of claims 29-42, wherein the second CAR polypeptide further comprises a hinge region.

44. The cell of claim 43, wherein the hinge region is located between the antigen binding domain and the transmembrane domain.

45. The cell of any one of claims 25-44, further comprising an altered α4β1 integrin.

46. The cell of claim 45, wherein the altered α4β1 integrin is mutated in the first exon.

47. The cell of any one of claims 45-46, wherein the altered α4β1 integrin cannot bind to vascular cell adhesion molecule 1 (VCAM1) on vascular endothelial cells.

48. The cell of any one of claims 45-46, wherein the altered α4β1 integrin has a frameshift mutation or deletion.

49. A CAR T cell comprising
a. a first CAR polypeptide comprising a LINGO1 antigen binding domain; and
b. a second CAR polypeptide comprising a MOG specific receptor and a Fas domain.

50. A CAR T cell comprising
a. a CAR polypeptide comprising a MOG specific receptor and a Fas domain; and
b. a mutated a4b1 integrin.

51. A CAR T cell comprising
a. a CAR polypeptide comprising a LINGO1 antigen binding domain; and
b. a mutated a4b1 integrin.

52. A CAR T cell comprising
a. a first CAR polypeptide comprising a LINGO1 antigen binding domain;
b. a second CAR polypeptide comprising a MOG specific receptor and a Fas domain; and
c. a mutated a4b1 integrin.

53. A nucleic acid sequence that encodes a CAR polypeptide, wherein the CAR polypeptide comprises a target specific receptor and a death domain.

54. The nucleic acid sequence of claim 53, wherein the CAR polypeptide is the CAR polypeptide of any one of claims 1-11.

55. A nucleic acid sequence that encodes a CAR polypeptide, wherein the CAR polypeptide comprises a LINGO1 antigen binding domain, a transmembrane domain, and an intracellular signaling domain.

56. The nucleic acid sequence of claim 55, wherein the CAR polypeptide is the CAR polypeptide of any one of claims 12-24.

57. A method of treating a subject having cancer comprising administering a composition comprising a CAR T cell to a subject having cancer,
wherein the CAR T cell comprises a CAR polypeptide comprising a LINGO1 antigen binding domain, a transmembrane domain, and an intracellular signaling domain,
wherein the subject having cancer has cancer cells expressing LINGO1,
wherein the CAR T cell binds LINGO1 on the cancer cells activating the CAR T cell to kill the cancer cell.

58. A method of treating a subject having cancer comprising administering a composition comprising a CAR T cell to a subject having cancer,
wherein the CAR T cell is one or more of those in claims 12-24, wherein the subject having cancer has cancer cells expressing LINGO1,
wherein the CAR T cell binds LINGO1 on the cancer cells activating the CAR T cell to kill the cancer cell.

59. A method of treating cancer comprising administering a composition comprising a CAR T cell to a subject having cancer, wherein the CAR T cell comprises
a. a first CAR polypeptide comprising an over-expressed antigen binding domain, a transmembrane domain, and an intracellular signaling domain; and
b. a second CAR polypeptide comprising a non-cancer specific antigen receptor and a death domain;
wherein when the CAR T cell binds a non-cancer specific antigen, the second CAR polypeptide activates killing of the CAR T cell.

60. The method of claim 59, wherein the cancer is Ewing's sarcoma.

61. The method of any one of claims 59-60, wherein the over-expressed antigen is LINGO1.

62. The method of any one of claims 59-61, wherein the non-cancer specific antigen is MOG.

63. The method of any one of claims 59-62, wherein the CAR T cell further comprises a mutated a4b1 integrin.

64. A method of treating Ewing's Sarcoma comprising administering a composition comprising a CAR T cell to a subject having Ewing's Sarcoma, wherein the CAR T cell comprises
a. a first CAR polypeptide comprising a LINGO1 antigen binding domain; and
b. a second CAR polypeptide comprising a MOG specific receptor and a Fas domain;
wherein upon crossing the blood brain barrier the MOG specific receptor binds MOG on neurons and activates killing of the CAR T cell.

65. A method of treating Ewing's Sarcoma comprising administering a composition comprising a CAR T cell to a subject having Ewing's Sarcoma, wherein the CAR T cell comprises
a. a first CAR polypeptide, wherein the first CAR polypeptide is one or more of the CAR polypeptides of claims 11-24; and
b. a second CAR polypeptide, wherein the first CAR polypeptide is one or more of the CAR polypeptides of claims 1-10;
wherein upon crossing the blood brain barrier the MOG specific receptor binds MOG on neurons and activates killing of the CAR T cell.

66. The method of any one of claims 64-65, wherein the CAR T cell further comprises a mutated a4b1 integrin.

67. A method of reducing migration of CAR T cells across the blood brain barrier (BBB) comprising administering a composition comprising a CAR T cell to a subject wherein the CAR T cell comprises a mutated a4b1 integrin.

68. The method of claim 67, wherein the CAR T cell further comprises one or more of the CAR polypeptides of claims 1-24.

69. A method of inducing apoptosis of a CAR T cell comprising administering a composition comprising a CAR T cell to a subject wherein the CAR T cell comprises
a CAR polypeptide comprising a target specific receptor and a death domain
wherein when the target specific antigen receptor of the CAR T cell binds the target, the death domain activates killing of the CAR T cell.

70. A method of inducing apoptosis of a CAR T cell comprising administering a composition comprising a CAR T cell to a subject wherein the CAR T cell comprises
a CAR polypeptide comprising a healthy cell specific antigen receptor and a death domain,
wherein when the healthy cell specific antigen receptor of the CAR T cell binds a healthy cell specific antigen on a healthy cell, the death domain activates killing of the CAR T cell.

71. The method of any one of claims 69-70, wherein the CAR T cell further comprises one or more CAR polypeptide of claims 11-24.

72. The method of any one of claims 69-71, wherein the CAR T cell further comprises an altered α4β1 integrin.

73. The cell of any one of claims 25-48, wherein c-Jun is overexpressed in the cell.

74. The CAR T cell of any one of claims 49-52, further comprising increased levels of c-Jun.

75. The nucleic acid sequence of any one of claims 53-56, further comprising a sequence that encodes c-Jun.
